(19) United States
(12) Patent Application Publication
(10) Pub. No.: US 2024/0091327 A1
(43) Pub. Date: Mar. 21, 2024

(54) ANTIGENS FOR DETECTING TOXOPLASMA INFECTION BY MONITORING CELLULAR IMMUNITY

(71) Applicant: Washington University, St. Louis, MO (US)

(72) Inventors: Laurence David Sibley, St. Louis, MO (US); Kevin M. Brown, St. Louis, MO (US); Qiuling Wang, St. Louis, MO (US); Iti Saraav, St. Louis, MO (US)

(21) Appl. No.: 18/464,051

(22) Filed: Sep. 8, 2023

Related U.S. Application Data
(62) Division of application No. 17/742,023, filed on May 11, 2022, now abandoned.
Application No. 17/742,023 is a division of application No. 16/347,510, filed on May 3, 2019, now Pat. No. 11,351,233.
(60) Provisional application No. 62/417,136, filed on Nov. 3, 2016.
(60) Provisional application No. 62/550,393, filed on Aug. 25, 2017.

(51) Int. Cl. A61K 39/00; A61K 39/002; A61M 5/315

(52) U.S. Cl. CPC A61K 39/0002; A61K 39/002; A61M 5/31531; A61K 2039/54; A61K 2039/572; A61K 2039/6087

(57) ABSTRACT

Antigens of Toxoplasma gondii that provide specific and strong delayed type hypersensitivity (DTH) immune response, or which stimulate IFN-γ secretion, are used for testing subjects for infection. Any skin testing format may be adapted for testing for the delayed type hypersensitivity, including a patch, a needle, or a prong. Presence of DTH indicates infection. Alternate methods of detecting a T cell response including monitoring IFN-γ secretion may be used.

### TECHNICAL FIELD OF THE INVENTION

This invention is related to the area of clinical testing. In particular, it relates to cellular immune responses including delayed type hypersensitivity reactions and cytokine release, or interferon gamma secretion assays, and their use in diagnosis of toxoplasmosis.

### REFERENCE TO A SEQUENCE LISTING

This application contains a Sequence Listing that has been submitted in ASCII format via Patent Center and is hereby incorporated by reference in its entirety. The ASCII copy, created Sep. 8, 2023, is named “016695_US_DIV2”, and is 85.500 bytes in size.

### BACKGROUND OF THE INVENTION

In Toxoplasma gondii, there are three main compartments, called dense granules (GRA proteins), rhoptries (ROP), and micronemes (MIC proteins), which release antigens into the extracellular milieu (Carruthers V B, Sibley L D. 1997. Sequential protein secretion from three distinct organelles of Toxoplasma gondii accompanies invasion of human fibroblasts. Eur J Cell Biol 73:114-123). Although both GRA and MIC compartments release antigens constitutively at low levels, micronemes can be stimulated to release large amounts of antigen in response to certain environmental cues, such as contact with host cells or other host factors (Carruthers V B, Giddings O K, Sibley L D. 1999. Secretion of micronemal proteins is associated with Toxoplasma invasion of host cells. Cell Microbiol 1:225-236; Carruthers V B, Sibley L D. 1997. Sequential protein secretion from three distinct organelles of Toxoplasma gondii accompanies invasion of human fibroblasts. Eur J Cell Biol 73:114-123; Carruthers V B, Sibley L D. 1999. Mobilization of intracellular calcium stimulates microneme discharge in Toxoplasma gondii. Mol Microbiol 31:421-428). Collectively, proteins that are released either constitutively or in a regulated fashion have been defined as “excretory secretory antigens (ESA).”

The ESA fraction is enriched in secretory microneme (MIC) proteins but also contains constitutively secreted dense granule (GRA) proteins. Several MIC and GRA proteins have been described. Previous studies have shown that MIC2, and its binding partner MIC2 associated protein (M2AP), are abundant components of ESA (Huynh M H, Barenau K E, Harper J M, Beatty W L, Sibley L D, Carruthers V B. 2003. Rapid invasion of host cells by Toxoplasma requires secretion of the MIC2-M2AP adhesive protein complex. EMBO J 22:2082-2090). Additionally, MIC5 (Brydges S D, Sherman G D, Nockemann S, Loyens A, Daubener W, Dubremetz J, Carruthers V B. 2000. Molecular characterization of TgMIC5, a proteolytically processed antigen secreted from the micronemes of Toxoplasma gondii. Mol Biochem Parasitol 111:51-66) and MIC10 (Hoff E F, Cook S H, Sherman G D, Harper J M, Ferguson D J, Dubremetz J F, Carruthers V B. 2001. Toxoplasma gondii: molecular cloning and characterization of a novel 18-kDa secretory antigen, TgMIC10. Exp Parasitol 97:77-88) have been studied as soluble micronemal proteins that are immunogenic. Several MIC proteins interact: for example MIC1, MIC4 and MIC6 form a complex involved in recognition of host carbohydrates (Friedrich N, Santos J M, Liu Y, Palma A S, Leon E, Saouros S, Kiso M, Blackman M J, Matthews S, Feizi T, Soldati-Favre D. J Biol Chem. 2010 285:2064-76, Blumenschein T M, Friedrich N, Childs R A, Saouros S, Carpenter E P, Campanero-Rhodes M A, Simpson P, Chai W, Koutroukides T, Blackman M J, Feizi T, Soldati-Favre D, Matthews S. EMBO J. 2007 26:2808-20) Gene deletions of MIC1 or MIC3 alone do not have a profound effect on invasion, but the double mutant is attenuated, indicating these proteins plan complementary roles (Moiré N, Dion S, Lebrun M, Dubremetz J F, Dimier-Poisson I. Exp Parasitol. 2009 123:111-7). MIC1 has been used in a variety of immunodiagnostic assays based on detection of antibodies that react to this protein (Holec L, Gasior A, Brillowska-Dabrowska A, Kur J. Exp Parasitol. 2008 119:1-6) or to hybrid proteins containing MIC1 and other parasite antigens (Holec-Gasior L, Ferra B, Drapala D. Clin Vaccine Immunol. 2012 19:1977-9). As well, MIC1 and MIC4 have been used in vaccination studies in mice (Lourenco E V, Bernardes E S, Silva N M, Mineo J R, Panunto-Castelo A, Roque-Barreira M C. Microbes Infect. 2006 8:1244-51). Other studies have shown that the secretory proteins GRA4, GRA6, and GRA7 are targets of the immune response (Mercier C, Cesbron-Delauw M F. 2015. Toxoplasma secretory granules: one population or more? Trends Parasitol 31:60-71).

Delayed type hypersensitivity (DTH) responses are driven by cellular immune responses to antigens (Black C A. 1999. Delayed type hypersensitivity: current theories with an historic perspective. Dermatol Online J 5:7). Typically a test antigen is injected in the skin of the ear, flank, or footpad and swelling measured 24-48 hr later (Allen I C. 2013. Delayed-type hypersensitivity models in mice. Methods Mol Biol 1031:101-107). The most well-known test uses tuberculin, an extract of purified protein derivative (PPD) from mycobacteria, which is used in a skin test for tuberculosis infection. The skin test is also the basis for many allergy testing protocols. Although previous studies have used skin testing of toxoplasmin in mice and hamsters based on swelling and redness, these assays have not proven to be that specific or sensitive (Frenkel J K. 1948. Dermal hypersensitivity to toxoplasma antigens (toxoplasmins). Proc Soc Exp Biol Med 68:634-639). Previous studies testing toxoplasmin, a skin test reaction elicited by ESA antigens, showed that it was sensitive and specific for detecting individuals in France that were chronically infected with T. gondii (Rougier D, Ambroise-Thomas P. 1985. Detection of toxoplasmic immunity by multipuncture skin test with excretory-secretory antigen. Lancet 2:121-123). In those studies, the ES antigen was made from culture supernatants, fixed with formalin, and then dialyzed with a 10 kDa filtration step. In subsequent studies, others have indicated that the active component in toxoplasmin is in the range of 10 kDa to 50 kDa based on filtration (Veprekova. 1978. Approximative molecular weight of the active component in toxoplasmin. Folia Parasitol (Praha) 25:273-275). It should be noted that proteins may undergo proteolytic processing or breakdown, so this size range does not necessarily indicate the size or identity of the full-length protein. Although these studies refined our knowledge of the active components of ESA, the active components remain undefined at the molecular level. Moreover, there is no way to produce the ESA fraction in large quantities such that it could be made into a commercial product.

Delayed type hypersensitivity reactions are predominately driven by CD4+ memory T cells that recognize antigen from a previous exposure (Mantoux Test as a model for a secondary immune response in humans. Vukmanovic-Stejic M, Reed J R, Lacy K E, Rustin M H, Akbar A N. Immunol Lett. 2006 10793-101). Upon recognition of their cognate antigen, these memory T cells expand and produce cytokines including interferon gamma (IFN-γ) tumor necrosis factor (TNF) and other chemokines. This initial reaction also results in recruitment of mononuclear (i.e. monocytes) cells and polymorphonuclear (i.e. PMNs) cells from circulation into the tissue. Although the conventional DTH test relies on monitoring induration, and redness that develop at the site of injection, more recent tests have been developed to directly monitor T cells responses to specific antigens. Typically these responses are monitored in circulating T cells obtained from the leukocyte fraction of whole blood. Leukocytes, including antigen-presenting cells and T cells, are mixed in vitro with antigens and the resulting responses monitored by production of IFN-γ or other cytokines. In some applications there are referred to as INFγ-release or IFN-γ-secretion assays, owing the fact that IFN-γ is the primary cytokine thought to drive the DTH response. The advantages of such tests is that they are more quantitative than the traditional skin test, they can be completed with a single office visit, and they often suffer less from cross-reaction to environmental antigens.

The enzyme-linked immunospot or ELISpot assay was originally developed for detecting B cells that were secreting antigen-specific antibodies (A solid phase immunoenzymatic technique for the enumeration of specific antibody-secreting cells. Sedgwick J D, Holt P G. J Immunol Methods. 1983 February 57:301-9). It has seen been modified to detect cytokines secreted by different immune cells. The principle of the assay is that it relies on a sandwich ELISA where a membrane-backed microplate (typically polyvinylidene difluoride) is coated with antibodies to a particular cytokine. Cells from healthy or immune donors are added the plate and incubated overnight in medium under standard culture conditions. Cytokines secreted during this incubation are captured by the antibody-coated membrane. Following the incubation period, the cells are washed off and the captured cytokine is detected by a second antibody that is specific for the protein of interest. Detection is accomplished using an enzyme-linked reagent, either secondary antibody, or streptavidin to detect the biotinylated primary antibody.

ELISpot assays have previously been used for detection of IFN-γ secretion by T cells in patients that were chronically infected with Toxoplasma gondii (Evolving characteristics of toxoplasmosis in patients infected with human immunodeficiency virus-1: clinical course and Toxoplasma gondii-specific immune responses. Hoffmann C, Ernst M, Meyer P, Wolf E, Rosenkranz T, Plettenberg A, Stoehr A, Horst H A, Marienfeld K, Lange C. Clin Microbiol Infect. 2007 13:510-5). This study focused on immunocompromised patients and used the ELISpot assay as a surrogate for CD4+ T cell responses to whole antigen. Although this study did not evaluate the ELISpot assay as a primary diagnostic tool, it suggests that the degree of immunity in a patient can be inferred from the strength of the response in the ELISpot assay. In this case the ELISpot test was conducted with whole parasite antigen and no attempt was made to define useful antigens that would increase sensitivity or specificity using this assay.

### SUMMARY OF THE INVENTION

According to one aspect of the invention, a Toxoplasma gondii-derived antigen composition is provided. The composition comprises a Toxoplasma gondii-derived antigen selected from the group consisting of: isolated and purified MIC1, MIC3, MIC4, or MIC6; truncated MIC1, MIC3, MIC4, or MIC6; extended MIC1, MIC3, MIC4, or MIC6; a fusion protein comprising any two or more of MIC1, MIC3, MIC4, or MIC6; a fusion protein of any of MIC1, MIC3, MIC4, or MIC6 with a moiety that enhances or facilitates purification, recombinant production, or immune cell stimulation, and combinations thereof as elements of an antigen or components of a composition. The Toxoplasma gondii-derived antigen composition may alternatively or additionally comprise or consist of any of the antigens shown in Table 1.

According to another aspect of the invention, a kit is provided. The kit comprises (a) a Toxoplasma gondii-derived antigen composition and (b) an applicator device for administration of the Toxoplasma gondii-derived antigen to a subject. The composition comprises a Toxoplasma gondii-derived antigen selected from the group consisting of: isolated and purified MIC1, MIC3, MIC4, or MIC6; truncated MIC1, MIC3, MIC4, or MIC6; extended MIC1, MIC3, MIC4, or MIC6; a fusion protein comprising any two or more of MIC1, MIC3, MIC4, or MIC6; a fusion protein of any of MIC1, MIC3, MIC4, or MIC6 with a moiety that enhances or facilitates purification, recombinant production, or immune cell stimulation, and combinations thereof as elements of an antigen or components of a composition. The Toxoplasma gondii-derived antigen composition may comprise or consist of any of the antigens shown in Table 1.

According to yet another aspect of the invention a method of delivering Toxoplasma gondii-derived antigen to a subject is provided. An applicator device that is loaded with a Toxoplasma gondii-derived antigen composition is contacted with skin of the subject. The Toxoplasma gondii-derived antigen composition is thereby delivered to the skin of the subject. The composition comprises a Toxoplasma gondii-derived antigen selected from the group consisting of: isolated and purified MIC1, MIC3, MIC4, or MIC6; truncated MIC1, MIC3, MIC4, or MIC6; extended MIC1, MIC3, MIC4, or MIC6; a fusion protein comprising any two or more of MIC1, MIC3, MIC4, or MIC6; a fusion protein of any of MIC1, MIC3, MIC4, or MIC6 with a moiety that enhances or facilitates purification, recombinant production, or immune cell stimulation, and combinations thereof as elements of an antigen or components of a composition. The Toxoplasma gondii-derived antigen composition may comprise or consist of any of the antigens shown in Table 1.

Yet another aspect of the invention is an applicator device for administering one or more Toxoplasma gondii-derived antigens to a mammal. The applicator device comprises one or more Toxoplasma gondii-derived antigens. The Toxoplasma gondii-derived antigens are selected from the group consisting of: isolated and purified MIC1, MIC3, MIC4, or MIC6; truncated MIC1, MIC3, MIC4, or MIC6; extended MIC1, MIC3, MIC4, or MIC6; a fusion protein comprising any two or more of MIC1, MIC3, MIC4, or MIC6; a fusion protein of any of MIC1, MIC3, MIC4, or MIC6 with a moiety that enhances or facilitates purification, recombinant production, or immune cell stimulation, and combinations thereof as elements of an antigen or components of a composition. The Toxoplasma gondii-derived antigen composition may comprise or consist of any of the antigens shown in Table 1.

Yet another aspect of the invention is a method of eliciting and/or monitoring a T cell response in a subject. A Toxoplasma gondii-derived antigen composition is contacted with T cells of the subject. The Toxoplasma gondii-derived antigen composition induces a T cell response, which may involve production or secretion of cytokines. The Toxoplasma gondii-derived antigen composition may be isolated and purified MIC1, MIC3, MIC4, or MIC6; truncated MIC1, MIC3, MIC4, or MIC6; extended MIC1, MIC3, MIC4, or MIC6; a fusion protein comprising any two or more of MIC1, MIC3, MIC4, or MIC6; a fusion protein of any of MIC1, MIC3, MIC4, or MIC6 with a moiety that enhances or facilitates purification, recombinant production, or immune cell stimulation, and combinations thereof as elements of an antigen or components of a composition. The Toxoplasma gondii-derived antigen composition may comprise or consist of any of the antigens shown in Table 1.

These and other embodiments which will be apparent to those of skill in the art upon reading the specification provide the art with

### BRIEF DESCRIPTION OF THE DRAWINGS

The patent or application file contains at least one drawing executed in color. Copies of this patent or patent application with color drawing(s) will be provided by the Office by request and payment of the necessary fee.

FIG. 1 shows a luciferase-based secretion assay. Secretion was monitored by release of the reporter protein MIC1-GLuc into the supernatant. Samples include cells that were treated with BAPTA-AM to block secretion (Inhibitor), cells treated only with buffer (Mock), and cells treated with BSA and zaparinast (Stimulated). These later samples contain the fraction that is referred to as ESA.

FIG. 2 shows an expression system for purification of fusion proteins. SUMO is a ubiquitin-like protein that is highly soluble, aiding in the expression of proteins in E. coli. Proteins can be cleaved by SUMO protease to release tag-free proteins of high purity. The SUMO-fusion protein and the SUMO protease are both tagged with six histidine residues that mediate binding to nickel, allowing one step purification by affinity chromatography.

FIG. 3A-B shows luminol responses in mice. FIG. 3A) Animals were injected with antigens in The ear pinne. At 48 hr after injection, animals were imaged after injection of luminol using a Xenogen IVIS 200 instrument. Animals were injected with either ESA or total antigen. Numbers indicate antigen amounts in micrograms. FIG. 3B) Quantification of the images shown in A. Data were processed using Living Image software.

FIG. 4 Monitoring of lipopolysaccharide (LPS) using the limulus amebocyte assay (LAL). After purification over polymixin B resin, the level of LPS as monitored by the LAL assay was reduced by >50 fold.

FIG. 5 shows ELISpot assay detecting IFN-γ produced by splenocytes from naive and T. gondii infected mice. Samples treated with PBS or ESA during in vitro culture. Con A serves as a non-specific positive control.

FIG. 6A-B shows ELISpot data for IFN-γ secretion by T cell in response to purified ESA proteins. FIG. 6A) Responses from C57/BL6 mice, FIG. 6B) Responses from Balb/C mice. Individual data points represent a result from one mouse, either uninfected (gray) or chronically infected (black). SFC indicates “spot forming cells” that were positive for INFγ secretion.

### DETAILED DESCRIPTION OF THE INVENTION

The inventors have developed a standardized, abundant test antigen composition for use in sensitively and specifically testing individuals for infection by Toxoplasma gondii. Antigens that cause a non-specific reaction (whether the subject has been infected or not) and antigens that cause a specific reaction (only in subject that has been infected) have been identified. The latter have been purified and cloned and modified to form test reagents. The former have been eliminated from test reagents.

The compositions of antigens preferably contain only antigens that cause a specific reaction and are devoid of antigens that cause a non-specific reaction. Such preparation may be made by any means known in the art, including isolation and purification from, e.g., natural sources, recombinant production, or synthetic production. Carriers for the antigens may be any standardly used, typically a carrier that does not itself cause a DTH reaction or inhibit a DTH reaction by a bona fide antigen. Non-limiting examples of excipients that may be used for the antigen compositions are sucrose, mannitol, trehalose, and Hemaccel™ (intravenous colloid). Buffers, salts, sugars, preservatives, isotonic saline solutions, phosphate-buffered saline, can also be used in the compositions. Additional components and excipients include water, polymers, fatty acid esters, parabens. Compositions may be stored as convenient, including without limitation as lyophilized samples, at about or below 4 degrees C., and at about or below −70 degrees C.

Compositions of antigens may be free of other ESA components such as dense granular proteins (GRA), other microneme proteins, or other components which lead to lower sensitivity and/or specificity. An isolated and purified preparation may be from T. gondii organisms, from a recombinant host cell, or from a synthetic in vitro reaction. The isolated and purified protein may comprise at least at least 1%, at least 5%, 10%, at least 20%, at least 25%, at least 30%, at least 40%, at least 50%, at least 60%, at least 70%, at least 75%, at least 80%, at least 85%, at least 90%, at least 95%, at least 97%, or at least 99% of the protein in a composition.

Testing for DTH may be used in order to prevent or detect congenital toxoplasmosis, for example by testing women before or during pregnancy, respectively. Primary infection of pregnant women may lead to abortion or severe neonatal malformation. Testing may also be used in immunocompromised patients, in whom a severe form of the disease may be fatal. Testing for DTH might also be performed in healthy adults to determine their infectious status prior to performing a medical procedure as a consequence of which they may become immunocompromised. Detection of infection may be critical in managing the disease. If a positive DTH test occurs, it may be desirable to follow it with a serum test. Because the two types of tests detect different immunological pathways and components, the two types of tests may give complementary information. Serum tests detect antibodies, whereas DTH tests detect cellular immune responses.

As an alternative, an in vitro reaction may be used to detect a T cell response. The in vitro reaction may be performed on any source of T cells, including whole blood, serum, plasma, and other tissue sources of T cells. The T cells are contacted with one or more of the Toxoplasma gondii-derived antigens or an antigen composition. If the T cells are reactive with the antigens or antigen composition they release a cytokine such as interferon-γ or other cytokines. The presence of interferon-γ or other released cytokine can be detected using any technique known in the art, including but not limited to an antibody or a series of antibodies. The antibodies may be labeled for detection. An antibody may be attached to an enzyme, such as horseradish peroxidase or alkaline phosphatase that produces colored products in the presence of appropriate substrates. An antibody may be fluorescently labeled, as an alternative. The in vitro reaction product may be captured on a solid support or assayed in the reaction fluid.

Kits may comprise an outer package to contain all components as well as optional inner packaging to contain individual components or combinations of components. Optional components include instructions for assembly and/or administration, information on side effects, expiry information, etc. Information may be provided in paper form, on a digital medium, or as an internet address to such information.

Applicators may be any type as is known in the art for administering an antigen to the skin of a subject and developing a DTH response. These include without limitation patches, needles, multi-needle assemblies, prongs, multi-prong assemblies. Antigens may be administered individually at separate locations or in combination at a single location.

Fusion proteins can be made using recombinant DNA technology to express two or more proteins or polypeptide portions of proteins as a single expression product. Any suitable technique known in the art for making and expressing such fusion proteins may be used. In some embodiments, a non-T. gondii protein is fused to a T. gondii protein. In other embodiments, two distinct T. gondii proteins are fused together.

Amounts of antigen composition that may be administered can be empirically determined, but may be between 0.1 and 50 ug, between 0.5 and 25 ug, or between 1 and 10 ug.

Suitable subjects include, but are not limited to, a human, a livestock animal, a companion animal, a lab animal, and a zoological animal. A subject may or may not be known to have a TDP43-mediated disorder. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the animal is a laboratory animal. Non-limiting examples of a laboratory animal may include rodents, canines, felines, and non-human primates. In another preferred embodiment, the subject is a human.

MIC1 is normally a 456 residue (amino acid) protein that is processed in the parasite remove the N-terminal 16 residues. This leaves a total size of 440 residues. In contrast to this native protein, the form we have expressed, termed here truncated MIC1, is from residues 20-340, for a total size of 320 amino acids. This region of the protein contains a micronemal adhesive repeat that has been shown to bind to host sialic acid residues [Garnett, J. A., et al., Detailed insights from microarray and crystallographic studies into carbohydrate recognition by microneme protein 1 (MIC1) of Toxoplasma gondii. Protein Sci, 2009. 18(9): p. 1935-47.]. We expressed a truncated version of the protein in order to make it soluble, a property that would distinguish it from the native molecule that also contains a C-terminal galectin domain [Saouros, S., et al., A novel galectin-like domain from Toxoplasma gondii micronemal protein 1 assists the folding, assembly, and transport of a cell adhesion complex. J Biol Chem, 2005. 280(46): p. 38583-91.]. It may, as a result, be recognized differently by the immune system.

MIC3 is normally a 383 residue (amino acids) protein that is processed in the parasite to remove the N-terminal 26 residues. This leaves a mature protein of 357 residues. We expressed a truncated form of MIC3 from residues 134 to 383, for a total size of 250 residues. We expressed a truncated version of the protein in order to make it soluble, a property that distinguishes it from the native molecule. The truncated form of MIC3 lacks most of the N-terminal lectin domain (residues 67-145) but contains the EGF repeats (residues 145-359) described previously (The Toxoplasma gondii protein MIC3 requires pro-peptide cleavage and dimerization to function as adhesin. Cérède O, Dubremetz J F, Bout D, Lebrun M. EMBO J. 2002 21:2526-36).

MIC4 is normally a 580 residue (amino acid) protein that is processed in the parasite to remove the N-terminal 25 amino acids. This leaves a mature protein of 555 amino acids. Biochemical studies have shown that the full length protein is further processed at the N-terminus between residues 57-58 (VT-SS) and by a C-terminal processing event to generate a 50 kDa and a 15 kDa products (The toxoplasma micronemal protein MIC4 is an adhesin composed of six conserved apple domains. Brecht S, Carruthers V B, Ferguson D J, Giddings O K, Wang G, Jakle U, Harper J M, Sibley L D, Soldati D. J Biol Chem. 2001 276:4119-27). We expressed a truncated form of MIC4 from residues 58 to 231, for a total size of 173 residues, a property that distinguishes it from the native molecule. The region of the protein that we expressed contains the first two Apple domains, but lacks the C-terminal Apple domains 5,6 that mediate binding to host cells (The toxoplasma micronemal protein MIC4 is an adhesin composed of six conserved apple domains. Brecht S, Carruthers V B, Ferguson D J, Giddings O K, Wang G, Jakle U, Harper J M, Sibley L D, Soldati D. J Biol Chem. 2001 276:4119-27).

MIC6 is a 349 residue (amino acid) protein that is processed in the parasite to remove the first 23 residues. This leaves a mature protein of 326 residues that was expressed as a recombinant protein in E. coli. This full-length form of the protein contains three EGF domains, a single acidic domain and a transmembrane domain near the C-terminus as described previously (Structural insights into microneme protein assembly reveal a new mode of EGF domain recognition. Sawmynaden K, Saouros S, Friedrich N, Marchant J, Simpson P, Bleijlevens B, Blackman M J, Soldati-Favre D, Matthews S. EMBO Rep. 2008 9:1149-55).

The mixture of ESA proteins, previously referred to as useful for a human skin test [Rougier, D. and P. Ambroise-Thomas, Detection of toxoplasmic immunity by multipuncture skin test with excretory-secretory antigen. Lancet, 1985. 2(8447): p. 121-3] may contain proteins that elicit non-specific responses. By removing these contaminants and focusing on proteins that only give positive responses in infected animals (and individuals) including MIC1, MIC3, MIC4, and MIC6, and truncated and/or fused forms of these proteins, our test achieves properties that are superior to the natural mixture of ESA proteins.

Previous studies have identified short peptide residues that enhance uptake by dendritic cells and increase the efficiency of antigen presentation [Sioud, M., et al., A novel peptide carrier for efficient targeting of antigens and nucleic acids to dendritic cells. FASEB J, 2013. 27(8): p. 3272-83.]. The receptor to which these peptides bind on host dendritic cells is not known. Nonetheless, it is likely that these short sequences work by enhancing uptake of the antigen and priming the presentation pathway. These steps of antigen uptake, processing, and presentation are critical for the DTH response. MIC1, MIC3, MIC4, and MIC6 and truncated and/or fused forms of these proteins, can be expressed so that these sequences are either at the N- or C-termini. These modified antigens can be purified under conditions that minimize contamination with LPS. Levels of LPS may be reduced to less than 0.5 EU/ml, less than 0.25 EU/ml, less than 0.1 EU/ml, less than 0.05 EU/ml. Any modification described herein for MIC1, 3, 4, or 6 can also be applied to any of the proteins of Table 1 or Table 2.

The above disclosure generally describes the present invention. All references disclosed herein are expressly incorporated by reference. A more complete understanding can be obtained by reference to the following specific examples that are provided herein for purposes of illustration only, and are not intended to limit the scope of the invention.

### EXAMPLES

### Example 1

### Stimulation of Microneme Secretion

Freshly isolated tachyzoites of Toxoplasma gondii were stimulated to secrete Excretory-Secretory Antigens (ESA) using procedures similar to those published previously [1]. In brief, high-binding 96-well plates were coated with 1% bovine serum albumin (BSA) in phosphate buffered saline (PBS) pH 7.4 or PBS alone at 4° C. overnight. The next day, plates were washed with PBS to remove soluble BSA just prior to the secretion assay. Freshly harvested type I strain RH-parasites were purified and resuspended in intracellular buffer (25 mM HEPES pH 7.4, 142 mM KCl, 5 mM NaCl, 1 mM MgCl₂, 2 mM EGTA, 5.6 mM D-Glucose). Control parasites were treated with 50 μM BAPTA-AM to suppress secretion and added to wells washed with PBS (no BSA coating). For stimulated samples, untreated parasites were added to BSA-coated wells in the presence of 500 μM zaprinast. Parasites were allowed to secrete for 10 min at 37° C. prior to collection of the supernatant. Duplicate samples for each treatment were processed for liquid chromatography and tandem mass spectrometry (LC/MS-MS).

### Identification of Toxoplasma-Secreted Proteins in ESA by LC-MS/MS

Secreted proteins were detected by LC-MS/MS as previously described [1]. In brief, ESA samples were reduced with 10 mM Tris-(2-carboxyethyl)phosphine and alkylated with 20 mM iodoacetamide before digestion overnight with 0.5 ug of trypsin. After desalting, the digest was then dried down and resuspended in 15 μl of 5% acetonitrile, 0.1% formic acid. Five microliters was resolved by LC-MS/MS on a NanoLC Ultra (Eksigent Technologies) coupled with an LTQ-Velos Pro Orbitrap (Thermo Scientific) using a 2 hr gradient. Raw data were processed and compared to the predicted proteome of the T. gondii genome to identify the protein components of ESA, as described previously [2]. For comparative semi-quantitative analysis, fold-enrichment was calculated from the frequency of spectral counts for peptides in the BSA-zaprinast stimulated samples compared to the BAPTA-AM treated control (set at 1 where no peptides were detected) for each protein detected in ESA. Proteins were considered candidate ESA proteins if the average fold enrichment was ≥4 in both of two independent experiments. Additionally, the cell-cycle specific expression profile for each candidate gene was analyzed to determine if it matched the profile of known microneme protein encoding genes [3]. Only proteins that fit both criteria (4 fold enriched and micronemal transcriptional profile) were considered authentic ESA-enriched proteins.

Cloning and Expression of T. gondii Antigens

Gene sequences encoding T. gondii microneme (MIC) and dense granule (GRA) proteins were obtained from ToxoDB (Protocol: http; Domain: toxodb; Top level domain: org). The coding regions were analyzed for the presence of predicted transmembrane or signal peptides using the ExPASy server (Protocol: https; subdomain: www; second level domain: expasy; Top level domain: org). Proteins were expressed either as full-length proteins or as truncations that were designed to eliminate undesirable hydrophobic regions. Genes were cloned from cDNA produced by Superscript III (Thermo Fischer Scientific) reverse transcriptase priming from polyA mRNA isolated from the type II ME49 strain, according to the manufacturer's recommendations. Primers to the coding regions of interest were designed to contain a BsaI site at the 5 end, and a XbaI site at the 3 end, in order to be compatible with the N-terminal His-tagged SUMO system from LifeSensor (pE-SUMO vector). PCR amplicons containing the region of interest were digested with BsaI and XbaI and cloned into similarly prepared pE-SUMO vector and transformed into competent XL1-Blue E. coli. Ampicillin resistant transformants were checked by PCR amplification and Sanger sequencing to verify plasmid inserts. To express recombinant proteins, pE-SUMO vectors containing inserts of T. gondii genes were transformed into BL21(DE3) Rosetta pLysS E. coli cells grown in terrific broth (TB) or Luria broth (LB). For induction of protein expression, cells were grown as a 5 ml overnight culture, then diluted 1:1,000 in fresh LB or TB and cultured at 37° C. for 4-6 hr followed by addition of IPTG (0.5-1.0 mM) and culture at 15° C. overnight. Protein expression and solubility were tested by lysis of the cell pellet in CelLytic B buffer (Sigma-Aldrich) and separation of pellet and supernatant fractions that were analyzed by SDA PAGE.

Proteins were purified using His-select nickel affinity columns (Sigma-Aldrich), and eluted in 200-300 mM imidazole, 50 mM sodium phosphate pH 8.0, 300 mM NaCl, according to the manufacturer's instructions. In cases where the SUMO tag was removed, proteins were first bound to nickel beads and then treated with purified SUMO protease U1P1 that was cloned as a N-terminal fusion in pET22b (Novagen) and separately produced in E. coli. The His-tagged SUMO and His-tagged protease were bound to the His-select nickel beads and the eluted fraction was analyzed for enrichment of the cleaved recombinant T. gondii protein. Proteins were checked for purity and concentration by SDS PAGE stained with Coomassie Blue or Syrpo Ruby. Proteins were dialyzed against 150 mM NaCl, 50 mM Tris-HCl, pH 8.0 and stored in aliquots at a concentration of 0.3 μg/μl at −80° C. until used.

The purified recombinant proteins elicited strong responses in the Limulus amebocyte assay (LAL), the gold standard for monitoring lipopolysaccharide (LPS). To alleviate this problem, we treated the recombinant proteins with polymyxin B resin, an antibiotic that binds LPS (Endotoxin removal from protein solutions. Petsch D, Anspach F B. J Biotechnol. 2000 Jan. 21; 76(2-3):97-119). Recombinant proteins were incubated with Polymyxin B agarose-endotoxin removal resin (Sigma, USA) for 6-12 hrs at 4° C. using gentle end over end mixing to achieve an endotoxin level <0.1 EU/mg. Then, the proteins were eluted using endotoxin-free buffer containing Tris-HCl at pH 6-8 depending on the protein. The amount of residual LPS in the proteins preparation were checked using a LAL assay kit (Pierce, USA) according to the manufacturer's instructions. Purified endotoxin-free proteins were then filter sterilized, quantified and stored at −80° C.

### Chronic Mouse Infections

Specific-pathogen-free mice were obtained from Jackson Laboratories and housed in the Animal Care Facilities at Washington University School of Medicine. Animals were housed and cared for according to the NIH Guide for the Care and Use of Laboratory Animals as approved by the Animal Studies Committee at Washington University.

Female C57/BL6 or Balb/C mice, age 8-12 weeks, were infected with the type II strains PRU or ME49 by i.p. needle inoculation of tachyzoites grown in vitro, using procedures described previously [4]. Alternatively, naïve animals were infected by oral feeding of 5-10 tissue cysts from chronically infected mice, as described previously [4]. To prevent accidental death during acute infection, mice infected with the ME49 strain were given sulfadiazine in the drinking water (0.1-0.2 g/L) 4-10 days post infection. Chronic infections were confirmed by serological analysis of serum obtained 30 days post infection, as described previously [4].

### In Vitro Assays for IFN-γ Secretion

To obtain T lymphocytes for ELISpot analysis, we isolated splenocytes from naïve and T. gondii chronically infected mice. Spleens were harvested and splenocytes were isolated by passage through a 70-μm-pore-size nylon cell strainer. Splenocytes were pelleted and red blood cells (RBCs) were removed using RBC lysis buffer (Biolegend, USA) for 5 min at 4° C. Splenocytes were then washed in sterile PBS and HBSS media (Corning, USA). Splenocytes were finally resuspended in CTL-media (CTL, USA) supplemented with 1% L-glutamine and 1× Pen-strep antibiotics.

ELISpot assays were conducting in 96 well plate format using splenocytes isolated as described above. Briefly, 2.5×10⁵ cells per well were plated in 96-well plate pre-coated with murine IFN-γ capture antibody (Immunospot, CTL, USA) and cultured for 24 hrs with media alone, purified recombinant proteins (2 μg/ml), a recall positive control-ESA (2 μg/ml), a T cell non-specific positive control Con A (2 μg/ml) and purified recombinant control protein-SUMO (2 μg/ml) at 37° C. with 5% CO₂. After washing and developing the plate according to the manufacturer's instructions, the antigen recall response was determined by counting the number of spots (IFN-γ producing cells) per well per treatment. The number of IFN-γ producing T-cells following stimulation with T. gondii antigens, were detected and calculated using an ELISpot reader (Immunospot®S6 Core, CTL, USA).

### Antigen Injection and Monitoring of Luminol Reaction

Control or chronically T. gondii infected mice were used to test the delayed type hypersensitivity (DTH) response using a previously published protocol for monitoring luminol fluorescence after in vivo injection [5]. Control or chronically infected mice were injected with PBS, ESA proteins (1.5 ug/in 10 μL injection volume). Animals were injected s.c. either in the pinne of the ear (using PBS control on one side and antigen on the other) or s.c. in the back of the animal after it had been shaved to remove fur. At 24, 48, or 72 hr post injection, mice were anesthetized using isoflurane, injected i.p. with luminol (10 μL/gram of body weight of a 20 mg/ml stock) and imaged using an IVSI Spectrum in vivo Imaging System with exposure settings of 1-3 sec. Data were analyzed using the IVIS Living Image software to determine the relative light emission for the region where antigen was injected, compared to a neutral background region or to the PBS control injection. Data were graphed and analyzed using Prism (GraphPad).

MIC1 Predicted Protein Sequence Type II ME49:

(SEQ ID NO: 1)
MGQALFLTVLLPVLFGVGPEAYGEASHSHSPASGRYIQQMLDQRCQEIAA
ELCQGGLRKMCVPSSRIVARNAVGITHQNTLEWRCFDTASLLESNQENNG
VNCVDDCGHTIPCPGGVHRQNSNHATRHEILSKLVEEGVQRFCSPYQASA
NKYCNDKFPGTIARRSKGFGNNVEVAWRCYEKASLLYSVYAECASNCGTT
WYCPGGRRGTSTELDKRHYTEEEGIRQAIGSVDSPCSEVEVCLPKDENPP
VCLDESGQISRTGGGPPSQPPEMQQPADRSDERGGGKEQSPGGEAQPDHP
TKGGNIDLPEKSTSPEKTPKTEIHGDSTKATLEEGQQLTLTFISTKLDVA
VGSCHSLVANFLDGFLKFQTGSNSAFDVVEVEEPAGPAVLTIGLGHKGRL
AVVLDYTRLNAALGSAAYVV EDSGCSSSEEVSFQGVGSGATLVVTTLGE
SPTAVSA

The Form of MIC1 Used in the Assay (in Bold Above):

(SEQ ID NO: 2)
His-SUMO-(M)AYGEASHSHSPASGRYIQQMLDQRCQEIAAELCQGGLR
KMCVPSSRIVARNAVGITHQNTLEWRCFDTASLLESNQENNGVNCVDDCG
HTIPCPGGVHRQNSNHATRHEILSKLVEEGVQRFCSPYQASANKYCNDKF
PGTIARRSKGFGNNVEVAWRCYEKASLLYSVYAECASNCGTTWYCPGGRR
GTSTELDKRHYTEEEGIRQAIGSVDSPCSEVEVCLPKDENPPVCLDESGQ
ISRTGGGPPSQPPEMQQPADRSDERGGGKEQSPGGEAQPDHPTKGGNIDL
PEKSTSPEKTPKTEIHGDSTKATLEEGQQLTL

MIC1 Coding Sequence (Introns Spliced Out, Coding Region in Bold) Type II ME49:

(SEQ ID NO: 3)
acctgaaagcgggtgccgcgtcgctaccgtttcctgtggcgtctctagtg
cgacatccgaagtaacagtaacgtccggcatggaacgccgacgcgggtgt
tccagtcgcctggctccttctactcgcacttcgatgttacgttccttatt
ggtgcgacgcggttctcgtgttgctagacgtcgcaccggctgaaagctgt
agaaaatttagttattttcctgtcagctagcttgcaggagtgcgtttttg
tgtgttggtttcgtctcacatggctgctgatctgttgatgcagctgtgta
cacgtgcctcgattctgtagttgacctagaacggatttgcaaagATGGGC
CAGGCGTTGTTTCTCACCGTTCTATTGCCGGTGTTATTTGGCGTTGGGCC
AGAAGCATATGGAGAAGCGTCGCATTCTCATTCGCCGGCATCGGGACGTT
ATATACAACAGATGCTTGACCAACGCTGCCAAGAGATTGCTGCAGAACTC
TGCCAAGGCGGACTTCGTAAAATGTGTGTGCCCTCTAGCCGGATAGTAGC
TCGAAACGCCGTGGGCATTACTCATCAAAATACACTTGAATGGAGATGCT
TTGATACAGCCTCTTTGCTGGAGAGCAATCAAGAAAACAACGGTGTTAAT
TGCGTGGACGACTGTGGCCACACGATACCGTGTCCTGGCGGCGTACACCG
GCAAAACAGTAATCACGCAACGCGCCATGAGATACTGTCCAAATTGGTCG
AAGAAGGAGTACAACGGTTCTGCAGTCCTTATCAAGCATCTGCCAACAAG
TACTGTAACGACAAATTTCCAGGGACCATTGCGAGGAGGTCGAAGGGCTT
CGGAAACAATGTCGAGGTTGCGTGGAGGTGTTACGAGAAGGCCAGCTTGC
TGTACTCGGTTTATGCTGAGTGTGCGAGCAACTGCGGAACAACGTGGTAC
TGCCCTGGAGGACGACGAGGGACGTCGACAGAACTAGACAAGCGGCATTA
TACAGAAGAGGAAGGAATTCGCCAGGCAATCGGATCCGTCGACAGCCCAT
GTTCTGAAGTTGAAGTCTGCCTACCGAAGGATGAGAATCCCCCGGTGTGT
TTAGATGAAAGTGGCCAGATTTCACGAACTGGTGGTGGGCCACCGTCACA
ACCGCCTGAGATGCAACAGCCCGCCGATCGTTCGGACGAGAGAGGTGGCG
GTAAGGAACAGTCGCCTGGAGGAGAAGCTCAGCCGGACCATCCAACGAAG
GGTGGTAACATAGACCTGCCTGAGAAATCAACATCTCCCGAGAAGACGCC
GAAAACCGAGATCCATGGTGACAGCACGAAAGCGACGCTCGAAGAGGGGC
AGCAACTAACGCTCACGTTTATCTCCACTAAACTGGATGTTGCTGTAGGC
TCGTGTCATTCACTCGTCGCGAATTTCCTTGATGGATTTTTGAAGTTTCA
GACGGGCTCAAATTCGGCGTTCGATGTGGTAGAAGTGGAAGAGCCAGCAG
GACCCGCAGTGCTTACGATAGGTCTGGGACACAAAGGCCGTCTCGCTGTT
GTCCTCGACTACACCAGGCTCAATGCTGCTTTAGGATCAGCTGCTTACGT
GGTCGAAGATTCTGGATGCAGCTCAAGTGAAGAGGTTAGTTTCCAAGGAG
TGGGTAGTGGAGCGACGCTCGTGGTGACGACGCTTGGCGAGAGTCCTACG
GCCGTCTCTGCTTGAtttatagtactctttggagcatgcttgtggaggaa
cgggacaatctcggcaaaatcaggatgaagtttgtgagatacagatcgtt
cctgaacagtggaagatgcgtcactattacacctatatgcgtcctggttc
ttgtagagttggagttcttgcaggtgtaatgactatgacatacggatata
acttcatacggggaactgtg

Primers Used for Cloning

MIC1(20-340) Bsa1-F:
(SEQ ID NO: 4)
ACTGTGGTCTCTAGGTATGGAAGCATATGGAGAAGCGTCGCATTCTCA
MIC2(20-340) XBA1-R:
(SEQ ID NO: 5)
ACTGTTCTAGATCAGAGCGTTAGTTGCTGCCCCTCTTCGAGCGTCGCTT

MIC3 Predicted Protein Sequence Type II ME49:

(SEQ ID NO: 50)
MRGGTSALLHALTFSGAVWMCTPAEALPIQKSVQLGSFDKVVPSREVVSE
SLAPSFAVTETHSSVQSPSKQETQLCAISSEGKPCRNRQLHTDNGYFIGA
SCPKSACCSKTMCGPGGCGEFCSSNWIFCSSSLIYHPDKSYGGDCSCEKQ
GHRCDKNAECVENLDAGGGVHCKCKDGFVGTGLTCSEDPCSKRGNAKCGP
NGTCIVVDSVSYTCTCGDGETLVNLPEGGQGCKRTGCHAFRENCSPGRCI
DDASHENGYTCECPTGYSREVTSKAEESCVEGVEVTLAEKCEKEFGISAS
SCKCDNGYSGSASATSHHGKGESGSEGSLSEKMNIVFKCPSGYHPRYHAH
TVTCEKIKHFALDGAGNHDTTTYVARRRYPASL

The Form of MIC3 Used in the Assay (in Bold Above):

(SEQ ID NO: 51)
YHPDKSYGGDCSCEKQGHRCDKNAECVENLDAGGGVHCKCKDGFVGTGL
TCSEDPCSKRGNAKCGPNGTCIVVDSVSYTCTCGDGETLVNLPEGGQGC
KRTGCHAFRENCSPGRCIDDASHENGYTCECPTGYSREVTSKAEESCVE
GVEVTLAEKCEKEFGISASSCKCDNGYSGSASATSHHGKGESGSEGSLS
EKMNIVEKCPSGYHPRYHAHTVTCEKIKHFALDGAGNHDTTTYVARRRY
PASL

Coding Sequence (Introns Spliced Out, Coding Region Bold) Type II ME49:

(SEQ ID NO: 52)
tcttctcttcttccgtacttttccctgcatttcacacccctggtatgac
tccacaccgcgtgtaaatgtcccttaggtgacacccgcagcagcgcgta
ggaggaagtagatgtcagtgtagacgtttttgagatgagagacgataac
gtaaaatgccgccgataacttctgcattatacacactctctctccacgc
ctaggatgacaggtacggcggcacacggaggaaagtggggggggggggg
ggggcgaacagaaaggtcacatggaaggccgctcgactctccactcacg
aagtgaaggcttcgtcccgttttgctggacaacgaatgcgaacttcttc
actcgcttgtgacacacacaactccagaggcacagagatgtgaagcaga
agagtggcgtgtgcgtcgcttctgtcggcggcaagccccgctccgtctc
tttggtggcgattctggtgtgcaccgtgtgccaagaagttgcgtgtcac
gcgacttttggaaatgcatcaggttcagagtcgttatgttgcgattcag
gctctcggcagagaatcatttccctgtaagctagttgaactcgcctttt
taaaagcggcagcagtgcccttgtggaaggcctcactgtgcctactttc
ctcgtcctgagtttttccgccttcggcctcattttttgctcaccaaaat
cgtgtcctaccgtcaagttttgccatagactcctacgggaaaaaacaag
ccggtcgacacggacgacgcccgcagggaagcgtcccctccgcagaaat
cgggagacaactgtcgttgacggtgctgcgcgaaaggtcacagagtttc
cagtgtgttcatcagacctcactgtgcactgttagcggccgctgtcccg
cctggtcaacaagtatcacaccctcgtccccgccattggcacggagctc
gatgagctgcagtgtcgcttttaggggagtcgtgcaatcacgccgcaac
acaggcgtgattcgatcttcaattgctaggtaaccactcgtgcttggta
gctctgcaatggctcgagcgacgggggtgatgcaacatgctgctaaaaa
ctcgacagacgtgtcaccggaacccacctaaataggagaccacgggtct
ctggtgtgtcgcgtcgcattctcgcgtcgcattctcgcgtcgcaatgac
cggccagttgctcgacgtcgccagccgggactgaagagcgttcatcgag
tcagcagcattgcgtccccttgctcggtgaaaaaagactctctggtcga
gtctagctcgtgtcacttctgtttctaacctccttcgttcaccggtaca
cctccgatgtgacttttggtacacttgccctgtcgcacgacgcacgctg
tcactcaacttgctgctagcgcaatcgataggttccctcgaaccagcca
tcacacacacaccttttccgggaagacgtttgcgggcggtgggtcgcag
ctcgtcgagagtgcgtttctgtgcatttctgtgggcagtgcagcgcgtt
tgcgcgccttactctgtgtgtaacttccttgtccaacactggtaaaaAT
GCGAGGCGGGACGTCCGCGCTGTTGCACGCGCTCACCTTCAGTGGGGCC
GTGTGGATGTGCACCCCAGCGGAGGCTTTGCCGATTCAGAAGTCTGTGC
AGCTGGGCAGCTTTGACAAAGTTGTGCCGAGCCGCGAAGTCGTCTCTGA
GAGTCTTGCTCCGTCTTTCGCGGTGACTGAGACTCACTCGTCTGTGCAA
TCCCCCAGCAAGCAGGAGACGCAACTCTGTGCTATCTCGAGTGAAGGCA
AGCCATGTCGAAACCGTCAGTTGCACACTGACAACGGGTACTTCATCGG
GGCCAGTTGCCCCAAGAGCGCTTGCTGCAGCAAGACCATGTGCGGCCCC
GGCGGCTGCGGAGAATTCTGCTCCAGCAACTGGATTTTTTGCAGCAGTT
CGCTCATCTACCATCCTGACAAAAGCTATGGAGGAGACTGCAGCTGTGA
AAAGCAGGGCCATCGGTGCGACAAAAACGCAGAATGCGTCGAAAACTTG
GACGCGGGTGGGGGTGTGCACTGCAAGTGCAAAGACGGCTTCGTCGGCA
CTGGGTTGACTTGCTCCGAGGATCCTTGTTCAAAAAGAGGGAACGCGAA
GTGCGGACCCAACGGGACGTGCATCGTCGTCGATTCAGTCAGCTACACA
TGCACCTGCGGCGACGGCGAAACTCTAGTGAACCTCCCGGAAGGGGGAC
AAGGATGCAAGAGGACTGGATGTCATGCCTTCAGGGAGAACTGCAGCCC
TGGTAGATGTATTGATGACGCCTCGCATGAGAATGGCTACACCTGCGAG
TGCCCCACAGGGTACTCACGTGAGGTGACTTCCAAGGCGGAGGAGTCGT
GTGTGGAAGGAGTCGAAGTCACGCTGGCTGAGAAATGCGAGAAGGAATT
CGGCATCAGCGCGTCATCCTGCAAATGCGATAACGGATACTCCGGATCT
GCTTCCGCAACCTCCCACCATGGGAAAGGAGAATCGGGATCCGAGGGGA
GCTTGAGTGAAAAAATGAATATTGTCTTCAAGTGCCCCAGTGGCTACCA
TCCAAGATACCATGCCCACACCGTGACGTGTGAGAAAATTAAGCACTTT
GCCCTTGACGGGGCCGGCAACCACGACACGACTACGTATGTCGCAAGAC
GAAGGTACCCAGCGAGTCTCTGAgagcggagatcagcgcaaagacaaga
tgcagagtttgactcgagaaacaatagtaacacgaagtaaaaagtctcc
acactaagccaaggattgagaatatttcgatttgtgccgctggcaatag
tggccttggcctagaaagaagttctgcaacgaagcgatcggctcacacg
cggatacacagatgggtttgtaccgagaacgttaggtttgtgaaccgag
ttcaggtaaaacaaagtagattgtgcctttacgcagacagcgagggaaa
acatgaggacacactgccaactaaagcaagactgcctcactaattacca
ccgacacacgacatggttacccccgcgttttgccgcgtgcaaagtttga
attctgatggttctcgagtctgaaagcctaaaccgcccaaccatgtatg
aaataagaacccatcaaacgtgagacatctctgccgaagtgcctacgaa
aagaacgcttctgccactaggaggtgcggcctcttcattctatgagaac
ctgctttgtcggtgtcaacctctggggaaatcgcctgcctttacacatt
ttgctcgttgtagagcaagggatctgttgctgcgtttactccaatacaa
tgatcgccgtttcgctgtaggcaagcgatccgaaaatgtacgttcgagt
cagcagctacttgagaagcagccaacgccgacacttgctgcgtttgact
gaggtgcactcgcaaacagtctcgtctccccggggcaatttctgagaga
aatgcgggaatggacgtaatggtgctcttctgtgagtgctcttccacca
atttttcgacaagtgttttcgtgacagtcgagtataccttcttatgtca
ttctgtctccgtcagtgctatcggattcttcctattcctctaccctttc
tacagtcgcatacaaagctgctgaaacaagacttcctttgtctagggta
gttgtacactccacacatatctgactgaaacctacggcaggaagtctgg
tcggcactgtgcttccttgttggcttttcgtcgtttctttgtctacgag
cttcactgggtccttgacacggcttgtgagcgttgtgctcaatattcga
ccagctgtatttgtg

Primers Used for Cloning:

MIC3F (134-383)-BsaI-F
(SEQ ID NO: 53)
ACTGTGGTCTCTAGGTATGATCTACCATCCTGACAAAAGCTATGGAGGAG
ACT
MIC3F (64-383)-BsaI-R
(SEQ ID NO: 54)
ACTTGTTCTAGATCAGAGACTCGCTGGGTACCTTCGTCT

MIC4 Predicted Protein Sequence Type II ME49:

(SEQ ID NO: 55)
MRASLPVHLVVCTQLSAVWFGVAKAHGGHRLEPHVPGFLQGFTDITPAG
DDVSANVTSSEPAKLDLSCVHSDNKGSRAPTIGEPVPDVSLEQCAAQCK
AVDGCTHFTYNDDSKMCHVKEGKPDLYDLTGGKTASRSCDRSCFEQHVS
YEGAPDVMTAMVTSQSADCQAACAADPSCEIFTYNEHDQKCTFKGRGFS
AFKERGVLGVTSGPKQFCDEGGKLTQEEMEDQISGCIQLSDVGSMTADL
EEPMEADSVGACMERCRCDGRCTHETENDNTRMCYLKGDKMQLYSSPGD
RTGPKSCDSSCFSNGVSYVDDPATDVETVFEISHPIYCQVICAANPLCT
VFQWYASEAKCVVKRKGFYKHRKTGVTGVTVGPREFCDEGGSIRDREEA
DAVGSDDGLNAEATMANSPDFHDEVECVHTGNIGSKAQTIGEVKRASSL
SECRARCQAEKECSHYTYNVKSGLCYPKRGKPQFYKYLGDMTGSRTCDT
SCLRRGVDYSQGPEVGKPWYSTLPTDCQVACDAEDACLVFTWDSATSRC
YLIGSGESAHRRNDVDGVVSGPYTECDNGENLQVLEAKDTE

The Form of MIC4 Used in the Assay (in Bold Above):

(SEQ ID NO: 56)
SEPAKLDLSCVHSDNKGSRAPTIGEPVPDVSLEQCAAQCKAVDGCTHET
YNDDSKMCHVKEGKPDLYDLTGGKTASRSCDRSCFEQHVSYEGAPDVMT
AMVTSQSADCQAACAADPSCEIFTYNEHDQKCTFKGRGESAFKERGVLG
VTSGPKQFCDEGGKLTQEEMEDQISG

Coding Sequence (Introns Spliced Out, Coding Region Bold) Type II ME49:

(SEQ ID NO: 57)
ttttctgtgcatctgtgctgcaaaacgggcctctgtgcattatttcccc
accaacaattgccgcgtcgatccgggtcccgctcaagctctgcagaact
aggctctcgatatagatcagtacaatcattcgcttctgacaatcgcatc
gactgagcgacgcgttgatcgtcgactgtcgtgcgtcgcattcgggcat
ctcgaaccggtgttgattccctgtgtcattatttcacttccgtccttct
ctcgtggcgatctataatacgcgtgtgttgttgcgtgcattgcttgtgt
tgttgtggatgtgttttcttttgtgaccgctcacgaacaccccacgcaa
aATGAGAGCGTCGCTCCCGGTTCACCTCGTTGTGTGCACGCAGCTAAGT
GCCGTTTGGTTTGGAGTGGCTAAAGCCCATGGTGGACACCGACTGGAAC
CGCATGTTCCCGGATTCCTGCAAGGCTTCACTGATATCACGCCTGCAGG
TGATGACGTTAGTGCCAACGTAACAAGTTCGGAGCCTGCAAAACTTGAT
CTCTCTTGTGTGCACTCTGACAATAAGGGATCAAGGGCTCCCACAATAG
GCGAGCCAGTGCCAGATGTGTCCCTGGAACAATGTGCTGCGCAATGCAA
GGCTGTTGATGGCTGCACACATTTCACTTATAATGACGATTCGAAGATG
TGCCATGTGAAGGAGGGAAAACCCGATTTATACGATCTCACAGGAGGCA
AAACAGCATCGCGCAGTTGCGATAGATCATGCTTCGAACAACACGTATC
GTATGAGGGAGCTCCTGACGTGATGACAGCGATGGTCACGAGCCAGTCA
GCGGACTGTCAGGCTGCGTGTGCGGCTGACCCGAGCTGCGAGATCTTCA
CTTATAACGAACACGACCAGAAATGTACTTTCAAAGGAAGGGGGTTTTC
TGCGTTTAAGGAACGAGGGGTGTTGGGTGTGACTTCCGGGCCGAAACAG
TTCTGCGATGAAGGCGGTAAATTAACTCAAGAGGAGATGGAAGATCAGA
TCAGTGGCTGCATTCAATTGAGTGACGTTGGATCAATGACTGCTGACCT
GGAGGAGCCTATGGAGGCTGATTCTGTTGGCGCTTGTATGGAACGGTGC
CGCTGTGATGGAAGATGCACGCACTTCACGTTCAACGATAATACTCGGA
TGTGCTACCTCAAAGGTGACAAGATGCAGTTGTACTCATCTCCAGGTGA
CAGAACCGGCCCAAAGAGCTGCGATTCAAGCTGCTTCTCGAACGGGGTT
TCTTACGTCGATGATCCGGCGACAGATGTTGAGACCGTATTCGAAATTT
CACACCCAATTTATTGTCAAGTAATCTGCGCCGCAAATCCGTTGTGTAC
AGTGTTTCAGTGGTATGCCTCCGAGGCAAAGTGCGTCGTCAAGAGAAAG
GGGTTTTACAAACACAGAAAAACAGGTGTCACGGGAGTCACAGTGGGCC
CTCGGGAGTTCTGCGATTTTGGCGGTAGCATCCGCGACCGAGAAGAGGC
AGACGCCGTTGGATCAGACGATGGCCTCAACGCGGAAGCAACTATGGCA
AATTCTCCTGATTTTCACGACGAAGTAGAATGCGTCCACACGGGCAACA
TTGGGTCAAAAGCACAAACCATTGGAGAAGTGAAACGCGCAAGTAGTTT
GAGTGAGTGCAGAGCCAGATGCCAAGCGGAGAAAGAATGCAGCCACTAC
ACTTACAATGTAAAATCCGGTTTGTGTTATCCAAAAAGAGGAAAGCCTC
AATTTTATAAGTATCTTGGCGACATGACGGGATCCAGAACATGTGATAC
AAGTTGCCTTAGGAGGGGAGTCGATTACTCACAGGGCCCTGAAGTAGGA
AAGCCTTGGTATTCTACGCTGCCGACAGACTGCCAAGTTGCATGCGACG
CTGAGGATGCTTGCCTGGTGTTCACCTGGGATTCGGCGACGTCACGATG
CTACCTCATCGGCTCAGGTTTCTCGGCACATCGACGGAACGACGTGGAT
GGCGTGGTATCTGGACCCTATACTTTCTGTGACAATGGCGAAAACCTTC
AGGTGCTTGAAGCGAAAGACACAGAATGAcccaggagggtgccagatac
tttgtgtgactgcgacatgcagtcatgtactcaaagtgttgtacatgga
caggaggactttttttttaagtcattgcagaggtgcgttttcggagcag
cactataactgcgtcagcgactaagcacgccacgtagctgaatgaaacg
cagccaccttcgtgtatgtatgcttcgttttttgtcgctgtgcagtttt
gaatcatttcccttatgggacatttctgaaaaatgctccccgttcgctt
gtagcactatgagaggggccgaagactgcaatggaggtagcgctgcgtt
gaaaagacgaggcgctacatttcgcgtagcgacaaggccgtgtagagtt
ttgcttttcgcgagacactgctctgagtgtcatatgcatcaaatgcagt
ggtagcacacagaggtgagaagaatgatcacctgcgggggaatggcttt
gctaaacaacaaggtcgctgtgtgactttacacaacgaaactactgtgg
tgagtgctcagttgagtgaaaagaaatgccgcgttatcgtgagttctgg
ttcggtggactttgccaccgtagtaaaactcaacctgtaacggaatgcc
cagttttactgctctctttaaagggcgtccacgttctctatattcaagc
tgtttacccacctgcgtttcggtgcatcgcgcgtgccacatcaaaaatc
caggtaacggtgcgggacctatgctacactttatatctctcagaaagca
tacacccactgattatggacaacgctgtggtcgcgttgtaccacaatgc
aggaatactcagttcaccttgcaagtgttctggtgttcattgcgtgtca
gaagtacacgaaaagagacttctttggcctccaagtgatacgtaaccgc
ggcagtcatgaacagagtcactcgtgcttctgaaacgcacgtcttctgt
acagagacagatgcagtgtgcatacaggaagcccctcgattgttgccgt
agcaggtagccagtagaagaaacaaagacacggt

Primers Used for Cloning:

MIC4(58-231)-BasI-F
(SEQ ID NO: 58)
ACTGTGGTCTCTAGGTATGAGTTCGGAGCCTGCAAAACTTGATCTCTCTT
GTGT
MIC4(58-231)-XBAI-R
(SEQ ID NO: 59)
ACTGTTCTAGATCAGCCACTGATATGATCTTCCATCTCCTCTTGAGT

MIC6 Predicted Protein Sequence Type II ME49:

(SEQ ID NO: 60)
MRLFRCCAAAVVAAESLLWLKNGSPFFAFLPGNGEIADNCSGNPCGGTAA
GTCINTPSGYDCRCEPGYVLGVENDQVTCMMPSGVPMANFVQLSEKPAAC
SSNPCGPEAAGTCNETNSGYICRCNQGYRISLDGTGNVTCIVRQESGCEE
NGCGPPDAVQSCRRLTGTAGRLCVCKENFIATIDASAHITCKRVPPHYRK
PPFEFGKGGHPVDSEPSKRQREDEGESREPESDSTEPGRDQERRTPLEES
QEPEGSTPDSQQSRGGSGSDSTESEEQGKEREEGSGHAGAIAGGVIGGLL
LLSAAGAGVAYMRKSGSGGGEEIEYERGIEAAEASEVEVLVDLDSKTWD

The Form of MIC6 Used in the Assay (in Bold Above):

His-SUMO
(SEQ ID NO: 61)
SPFFAFLPGNGEIADNCSGNPCGGTAAGTCINTPSGYDCRCEPGYVLGVE
NDQVTCMMPSGVPMANFVQLSEKPAACSSNPCGPEAAGTCNETNSGYICR
CNQGYRISLDGTGNVTCIVRQESGCEENGCGPPDAVQSCRRLTGTAGRLC
VCKENFIATIDASAHITCKRVPPHYRKPPFEFGKGGHPVDSEPSKRQRED
EGESREPESDSTEPGRDQERRTPLEESQEPEGSTPDSQQSRGGSGSDSTE
SEEQGKEREEGSGHAGAIAGGVIGGLLLLSAAGAGVAYMRKSGSGGGEEI
EYERGIEAAEASEVEVLVDLDSKTWD

Coding Sequence (Introns Spliced Out, Coding Region Bold) Type II ME49:

(SEQ ID NO: 62)
cagtccggagcacactcctacaataaacttgatacgtgtcattttgtgaa
acgacacagcacataaccactcggactgtctcacgaagctgtagggcgga
ttcaccaatgatctttcgcagccgatccaaaactacttgcccacttccgg
tgtacgtacatcgcgcgacatgagaggcattcattgttttccatagaaaa
cactactggacaaccattcggtagcgcacaagttgagcctctgacaaatc
tttcctcatcacgtgaatacacgctgcgtgattcgtcagtgactccactg
tggtctttaaccaccatcagagtcctgtaagcatcctttgtttccgttta
aaatgcctgccagatggcacgacgccgtctggttttgccggctttctccg
agtcctattagactttgatgccttacggcttttttttaagaatggttctt
ttgagatttgccgactttccagttccgccaccagacgctcctgttgaact
gccaccggcacgatgcagtattccgccacgaaaacgcgcaccgcaagctc
cgctaccattaaacgggtttcgtctgctttagatgtttccttccgcgtca
tcaaggcaaaagcattgccactgatgttaccgaagctttcccgccatgct
gcgcacaatgcccaatcttccgtcacggacctcttccggtaaccacctaa
aggaggattactgggcaacccaaaacgctgcaacaagaagcacagtccag
gtgtcgctagattcgagcctgcatggtcgttccgtagctccatacaacaa
ttctctgtgtgacggcgagaggagtaacgcgctagtgtgtgtcagcgacg
cggcagtcgatccgatcctgcaacaggcagaggtgtgtcgatgctcagtg
atgcgacggcgtatctgaagaggactgtagctccaccacgaccttcgtgg
gagcacgaagtgtactctgttgtcgtcggtctcgtatttttttgagttgt
gtacttcgctgcaagaggagggtgagattcgacatctgtgggcgtttggg
atcgtgatgacatcgactgtgctttgatatatgatgtgttttttttcgat
tggatgagcacattccagtaagcttcctgccgcgcgtctctgctATGAGG
CTCTTCCGGTGCTGTGCTGCGGCCGTTGTGGCGGCCGAATCGTTACTGTG
GCTGAAGAACGGCTCCCCGTTTTTTGCCTTTCTTCCTGGGAATGGAGAGA
TTGCAGACAACTGCTCTGGGAATCCATGCGGTGGCACCGCAGCTGGTACG
TGCATAAACACACCATCTGGATATGATTGCAGGTGCGAACCAGGCTACGT
TCTGGGCGTTGAAAATGACCAGGTCACGTGCATGATGCCCTCAGGTGTAC
CCATGGCTAATTTTGTACAGCTGTCGGAAAAGCCTGCAGCTTGCAGCTCA
AACCCTTGTGGACCTGAGGCAGCCGGCACCTGCAACGAGACAAACAGTGG
TTACATTTGCCGCTGTAATCAAGGCTACAGAATATCTCTCGACGGGACAG
GAAACGTGACATGTATTGTAAGACAGGAAAGCGGCTGTGAGGAAAACGGG
TGTGGGCCGCCAGATGCAGTACAGAGTTGCCGCCGACTAACAGGGACGGC
AGGTCGACTATGTGTATGCAAGGAAAACTTTATAGCGACAATCGACGCCA
GTGCCCATATCACCTGCAAGCGTGTGCCTCCCCATTATAGGAAGCCTCCC
TTCGAATTTGGCAAGGGAGGTCATCCTGTGGACTCAGAACCATCGAAACG
CCAGAGGGAAGATGAAGGTGAAAGTCGTGAGCCTGAAAGCGACTCAACAG
AACCGGGGAGAGATCAGGAAAGAAGAACACCACTTGAGGAAAGCCAGGAA
CCGGAAGGAAGCACCCCGGACAGTCAGCAGAGCCGAGGTGGTTCTGGTAG
CGACAGTACCGAGAGCGAGGAACAAGGAAAGGAGAGAGAGGAAGGAAGTG
GACATGCTGGTGCGATCGCTGGGGGAGTTATTGGAGGCCTGTTACTTCTG
AGCGCTGCCGGAGCGGGTGTTGCATACATGAGAAAGAGTGGGAGCGGTGG
AGGGGAGGAGATAGAATACGAGAGGGGTATCGAGGCTGCAGAGGCCAGTG
AAGTCGAAGTCCTCGTTGATTTGGATAGCAAAACATGGGATTAAcacgtt
ctcggctgagacttcacaatgtagggtgtcgctggcagatcagctgcaat
gcgagaggtgacgcgagtagtgagcaccgcttcttttaagcgcggacatt
gtgctcggtcttctgtcacccccgaatcaaaacacatgtatgataatagt
tcctgttgacttcccctgccgacaaagaactgctgtgtcgaggccggctt
ctgtgcactcatcccaaatgagatggactgatgttttagagacacctcat
cgccgacggaaaccatcagctcccagagaaactatgctgcgtcgtttttt
aggtgatctgttgcgtaatgcgcaccttcatatcatctgtgtgttgactg
tttggtcgttttccgtttagtcaaatgaatgcagtgaaatgcagggaatt
tagcagacaccgagaactgtcctcttgttctgtgcgcgagttgtttttaa
cgtatagcgatgcgtttgcacttgatattaccctaagccatcagtgggta
tttagaggagcccacaggtgatgggggtgatccctgtttcttgtcatttg
gcttgtagggttcgctggaactatctggtgtcacggaagagtggctttac
tgtctgtccccaaacgcaaggcatcagtgtaaccccgataggactctgga
gacttctgcttcactgccgcgttgcaattttcccgcgtcatgtggcaata
acggtaattccacgtgcacgccgcataccggatctttgctcccaggcttt
cttatgaggtcggcatacgtacagcggcggcgtacctccgctctagagaa
gaccggtccaaccgactttgaacagcatgcttgtgaatgagtgcttaaac
accctgaagtgatggtggaatgtagcagtctgggacggttgatgcgagga
tatcaccattagcatagactaccttgctctttagcgaggcgagacaactt
atttaggtagccatgaaacacctcgatagtatcaatgacgacgtgcggtt
caccaacttccgtcgctagcgcagaaaacagtcggaaacacaactcggtg
agcacctgaagtgtcagtacacattcgaccgtcgggacccgggattccgc
aagtggcacccgctggtccagtagcaggaacctagttcattcagtataac
agatttggggcggcaaagagcaatttgctcgacctaacgcttgc

Primers Used for Cloning:

MIC6-(24-349)-BasI-F
(SEQ ID NO: 63)
ACTGTGTGCTCTAGGTATGTCCCCGTTTTTTGCCTTTCTTCCTG
MIC6-(24-349)BasI-R
(SEQ ID NO: 64)
ACTGTTCTAGATTAATCCCATGTTTTGCTATCCAAATCA

SUMO Protein Sequence:

This sequence is present at the N-terminus of SUMO fusions

Coding Sequence for His Tagged SUMO (his-SUMO)

(SEQ ID NO: 16)
ATGGGTCATCACCATCATCATCACGGGTCCCTGCAGGACTCAGAAGTCAA
TCAAGAAGCTAAGCCAGAGGTCAAGCCAGAAGTCAAGCCTGAGACTCACA
TCAATTTAAAGGTGTCCGATGGATCTTCAGAGATCTTCTTCAAGATCAAA
AAGACCACTCCTTTAAGAAGGCTGATGGAAGCGTTCGCTAAAAGACAGGG
TAAGGAAATGGACTCCTTAAGATTCTTGTACGACGGTATTAGAATTCAAG
CTGATCAGGCCCCTGAAGATTTGGACATGGAGGATAACGATATTATTGAG
GCTCACCGCGAACAGATTGGAGGT

Predicted Protein Sequence for his-SUMO:

(SEQ ID NO: 17)
MGHHHHHHGSLQDSEVNQEAKPEVKPEVKPETHINLKVSDGSSEIFFKIK
KTTPLRRLMEAFAKRQGKEMDSLRFLYDGIRIQADQAPEDLDMEDNDIIE
AHREQIGG

### Example 2

We have developed an efficient assay where we can control the amplitude of antigen release pharmacologically. We compared constitutive secretion (mock treatment), to conditions where we block or enhance microneme secretion. T. gondii antigens released into the extracellular milieu are likely to stimulate both humoral and cell-mediated immunity. Identification of immunogenic proteins in ESA has led to improved diagnostic reagents for T. gondii infection.

### Collection of Excreted/Secreted Antigen (ESA)

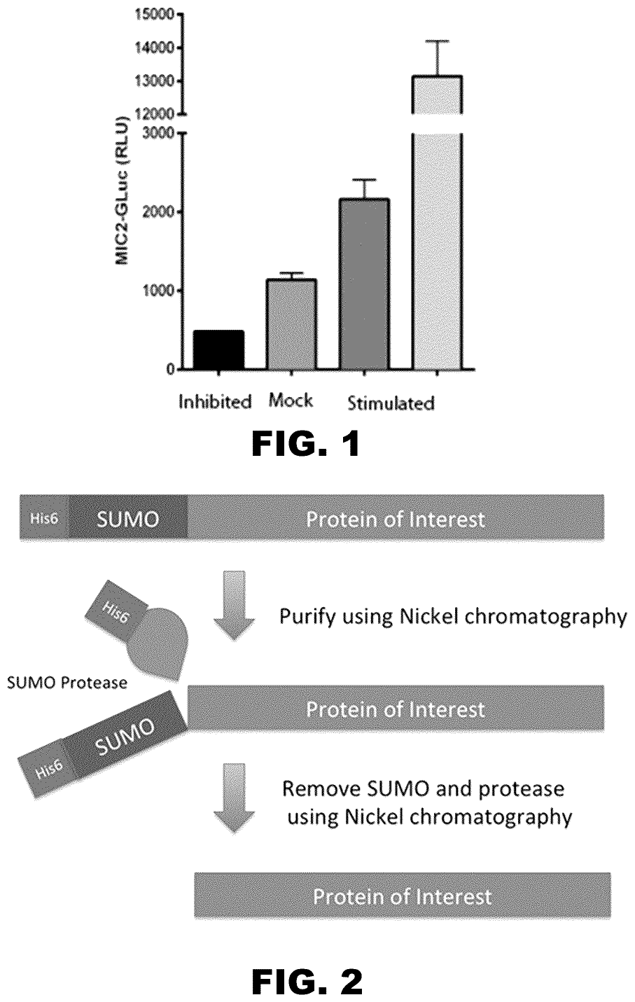

Toxoplasma gondii RH strain parasites were isolated from infected human fibroblasts, filtered, and washed extensively. Parasites were either left untreated (Mock), treated to block (Inhibited), or induce secretion of ES antigens (Stimulated). After 10 min at 37° C., the parasites were chilled to 4° C., centrifuged, and the cell-free supernatant was collected. To evaluate the complexity, samples were separated by SDS-PAGE and the protein composition was assessed by staining. In addition, we used a luciferase-based assay to detect a microneme reporter to specifically determine the level of microneme secretion in each of the fractions (FIG. 1).

### LC/MS-MS of ES Antigens

Samples were processed for mass spectrometry (MS), separated by LC, resolved on an Orbitrap MS/MS instrument, and analyzed using Mascot (Matrix Science, London, UK). Scaffold (Proteome Software Inc., Portland, OR) was used to analyze MS/MS peptides and establish protein identifications by comparison to gene databases.

We classified ES antigens based on enrichment of peptides in stimulated vs. blocked samples using a cutoff of 4-fold increase in two replicate samples. The proteins were further analyzed for their profile of expression during development to classify those that were bone fide micronemal proteins (the major component of ESA) vs. potential contaminants. The profile of micronemal proteins is highly characteristic and many of the secreted proteins share this transcriptional profile. Based on the fold enrichment and expression pattern, we generated a list of the most abundantly induced proteins in ESA (Table 1).

TABLE 1
Summary of ESA proteins identified by mass spectrometry.

| | | Fold |
|---|---|---|
| Gene ID | Product Description | Increase¹ |
| TGME49_267680 | microneme protein MIC12 (MIC12) | 39.0 |
| TGME49_291890 | microneme protein MIC1 (MIC1) | 34.7 |
| TGME49_294330 | EGF family domain-containing protein | 34.0 |
| TGME49_208030 | microneme protein MIC4 (MIC4) | 28.8 |
| TGME49_201780 | microneme protein MIC2 (MIC2) | 27.3 |
| TGME49_319560 | microneme protein MIC3 (MIC3) | 26.1 |
| TGME49_206510 | toxolysin TLN4 (TLN4) | 25.0 |
| TGME49_214940 | MIC2-associated protein M2AP | 21.5 |
| TGME49_234380 | hypothetical protein | 16.5 |
| | (TGME49_234380) | |
| TGME49_204050 | subtilisin SUB1 (SUB1) | 16.5 |
| TGME49_218520 | microneme protein MIC6 (MIC6) | 15.5 |
| TGME49_250710 | microneme protein MIC10 (MIC10) | 13.2 |
| TGME49_293440 | hypothetical protein | 13.0 |
| | (TGME49_293440) | |
| TGME49_232280 | hypothetical protein | 11.5 |
| | (TGME49_232280) | |
| TGME49_204130 | perforin-like protein PLP1 (PLP1) | 11.5 |
| TGME49_243930 | hypothetical protein | 5.5 |
| | (TGME49_243930) | |
| TGME49_277080 | microneme protein MIC5 (MIC5) | 5.4 |
| TGME49_258870 | hypothetical protein | 4.5 |
| | (TGME49_258870) | |

¹Increase of peptide spectral counts in stimulated fraction vs. control

### Example 3

To express these proteins recombinantly, we are using a fusion system based on the E. coli protein SUMO, which allows for production and purification of soluble, tagged proteins. From this group of initial candidates, we have successfully cloned, expressed, and purified all of the proteins shown in Table 2. These proteins were tested here as fusion proteins with SUMO as a control. However, they can also be purified away form SUMO after protease cleavage by nickel chromatography as shown in FIG. 2.

### Example 4

### Development of Model for DTH Using Bioluminescence

We tested an alternative method that relies on light production in the skin. The basis for this method is that recruitment of monocytes and neutrophils to the site of inflammation can be detected using luminol, a substrate that gives off light when converted by myeloperoxidase (Gross S, Gammon S T, Moss B L, Rauch D, Harding J, Heinecke J W, Ratner L, Piwnica-Worms D. 2009. Bioluminescence imaging of myeloperoxidase activity in vivo. Nat Med 15:455-461). This method has been shown to be sensitive for detecting DTH responses in the mouse and for monitoring leukocyte influx to sites of infiltration (Gross, supra).

### Example 5

### The Luminol DTH Response is Specific to ESA

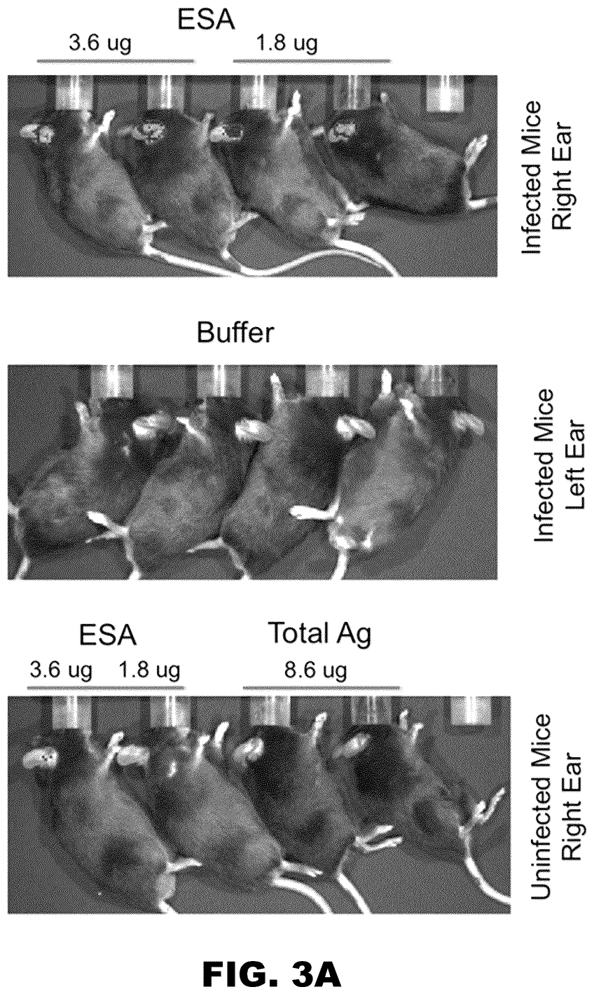

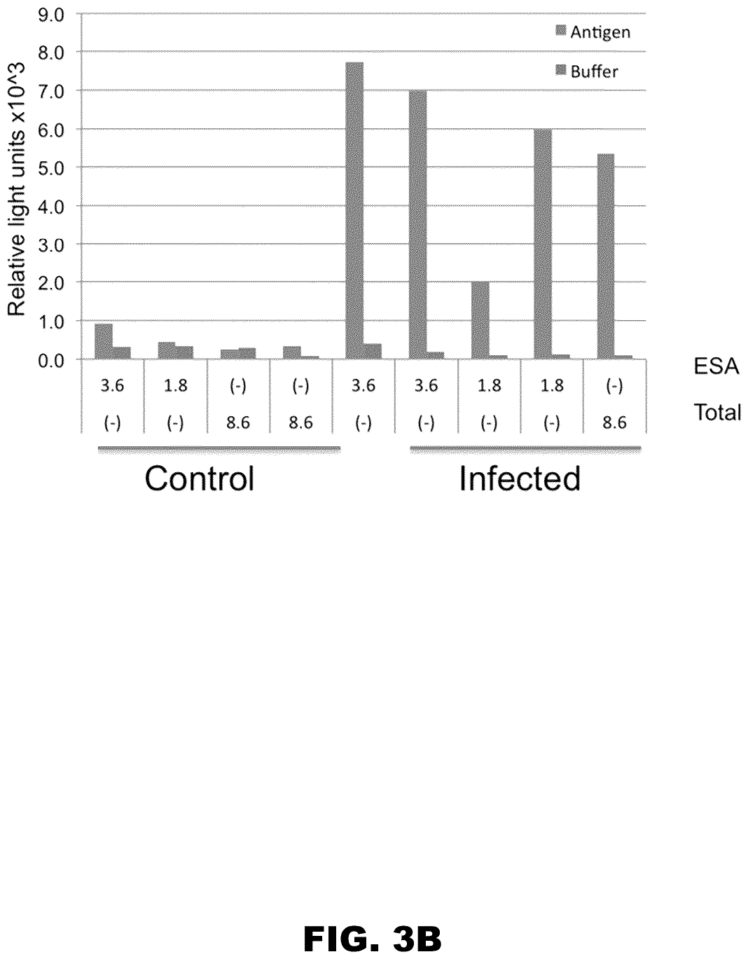

We have modified the luminol assay used for monitoring DTH responses in the mouse by injecting antigen in the pinne of the ear. In order to confirm that the DTH responses that we were detecting were in fact due to antigens in ESA, we compared the response for ESA to total parasite antigen or to PBS. The response detected by luminol was highly enriched in ESA sample compared to the PBS control or to total antigen (FIG. 3A, 3B). The response is also only seen in infected animals, confirming that it is due to a specific immune response. This experiment demonstrates that the DTH response is driven by antigens that are enriched in ESA.

### Example 6

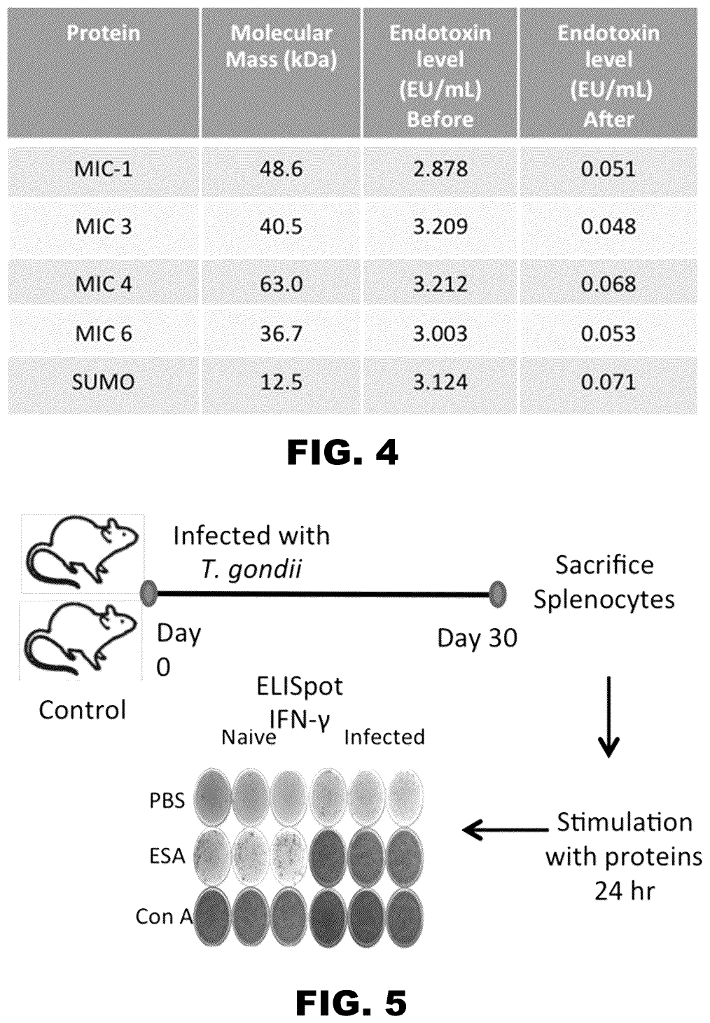

We have focused on the ESA antigens defined in Table 1 along with some constitutively secreted dense granule proteins (GRA) that have previously been shown to be immunogenic. We have cloned, expressed, and purified 12 proteins for testing, as shown in Table 2. Test proteins were purified as fusion proteins with SUMO, an E. coli protein that facilitates solubility. We have also purified the SUMO protein as a control. To avoid non-specific responses due to endotoxin (LPS) we purified ESA proteins using polymyxin B, a detergent like molecule that removes endotoxin. The resulting purified proteins showed reduced levels of LPS when examined using the limulus amebocyte assay (FIG. 4).

TABLE 2

| | Constructs | Clone | | Molecular |
|---|---|---|---|---|
| ESA Protein | (aa) | Vector | Strain | weight (kD) |
| MIC10 | Full length | pE- | Rosetta | 23.1 |
| | | SUMO | (DE3) | |
| GRA7 | Full length | pE- | Rosetta | 26 |
| | | SUMO | (DE3) | |
| GRA6 | Full length | pE- | Rosetta | 25 |
| | | SUMO | [DE3] | |
| M2AP | Full length | pE- | Rosetta | 34.6 |
| | | SUMO | (DE3) | |
| MIC5 | Full length | pE- | Rosetta | 19.9 |
| | | SUMO | [DE3] | |
| MIC6 | Full length | pE- | Rosetta | 36.7 |
| | | SUMO | (DE3) | |
| Hypothetic Protein 2 | Full length | pE- | Rosetta | 30.2 |
| (TGME49_232280) | | SUMO | (DE3) | |
| GRA4 | 21-247 | pE- | Rosetta | 38 |
| | | SUMO | (DE3) | |
| MIC1 | 20-340 | pE- | Rosetta | 48.6 |
| | | SUMO | (DE3) | |
| MIC3 | 134-383 | pE- | Rosetta | 40.5 |
| | | SUMO | (DE3) | |
| MIC4 | 58-231 | pE- | Rosetta | 63.0 |
| | | SUMO | (DE3] | |
| Hypothetic Protein 1 | 89-347 | pE- | Rosetta | 38.7 |
| (TGME.234380) | | SUMO | (DE3) | |

### Example 7

An in vitro method to monitor antigen presentation This method is based on the ability of specialized immune cells (dendritic cells and macrophages) to present antigen to memory T-cells that in turn produce interferon gamma (IFN-γ). T-cells that produce IFN-γ in response to recall antigens are one of the primary drivers of the DTH response (Black C A. 1999. Delayed type hypersensitivity: current theories with an historic perspective. Dermatol Online J 5:7). However, instead of injecting antigens into the skin, we monitored the production of IFN-γ using a technique called ELISpot to specifically detect IFN-γ producing T-cells following antigen presentation in vitro. Following incubation of splenocytes with specific antigens or controls, IFN-γ is captured by an antibody on the membrane and then detected using an enzyme-linked immuno-assay (the blue spots represent positives). As shown in FIG. 5, this assay measures robust responses of splenocytes from T. gondii infected mice incubated with ESA, while there is minimal response in naive animals. ConA is used as a non-specific stimulus as it evokes responses from all T-cells regardless of specific antigen presentation. We have adapted this assay for monitoring recombinant ESA proteins and peptides that are synthetically produced.

### Example 8

### Using the ELISpot Assay to Monitor IFN-γ Secretion to Individual ESA Proteins

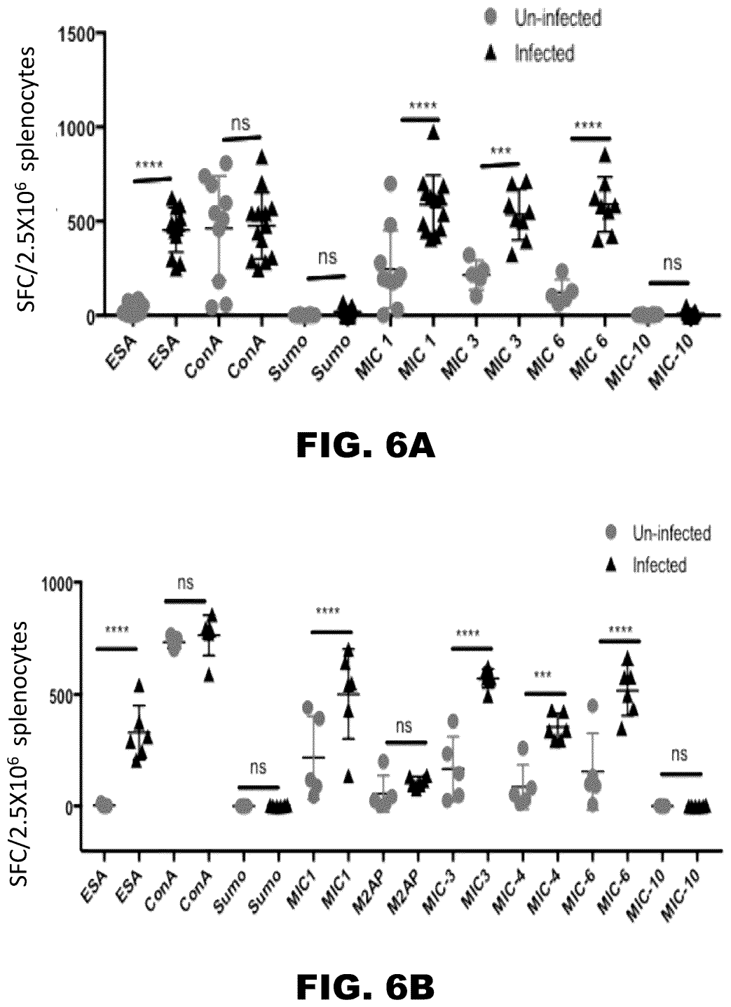

The ELISpot assay was also used to examine the response of uninfected and chronically infected mice to individual ESA proteins that were produced recombinantly. Initially, the ESA fraction was compared to ConA as a positive control and SUMO as a negative control. High numbers of spot forming cells (SFC) were detected using an IFN-γ specific ELISpot assay as shown in FIG. 6. Individual ESA proteins were used at 3 micrograms per well in combination with 10⁵ splenocytes. Specific responses were detected to MIC1, MIC3, MIC4 and MIC6, but not to M2AP or to MIC10 as shown in FIG. 6. Similar responses were seen in infected C57/BL6 mice (FIG. 6A) and Balb/C mice (FIG. 6B).

### REFERENCES

The disclosure of each reference cited below and throughout this application is expressly incorporated herein.

### REFERENCES FOR EXAMPLE 1

1 Brown, K. M., S. Lourido, and L. D. Sibley, Serum Albumin Stimulates Protein Kinase G-dependent Microneme Secretion in Toxoplasma gondii. J Biol Chem, 2016. 291(18): p. 9554-65.
2. Etheridge, R. D., et al., ROP 18 and ROP 17 kinase complexes synergize to control acute virulence of Toxoplasma in the mouse. Cell Host Microbe, 2014. 15: p. 537-550.
3. Behnke, M., et al., Coordinated progression through two subtranscriptions underlies the tachyzoite cycle of Toxoplasma gondii. Plos One, 2010. 5: p. e12354.
4. Khan, A., et al., Geographic separation of domestic and wild strains of Toxoplasma gondii in French Guiana correlates with a monomorphic version of chromosome 1a. Plos Negl. Trop. Dis., 2014. 8: p. e3182.
5. Gross, S., et al., Bioluminescence imaging of myeloperoxidase activity in vivo. Nat Med, 2009. 15(4): p. 455-61.

### REFERENCES FOR EXAMPLES 2-4

1. Carruthers V B, Sibley L D. 1997. Sequential protein secretion from three distinct organelles of Toxoplasma gondii accompanies invasion of human fibroblasts. Eur J Cell Biol 73:114-123.
2. Carruthers V B, Giddings O K, Sibley L D. 1999. Secretion of micronemal proteins is associated with Toxoplasma invasion of host cells. Cell Microbiol 1:225-236.
3. Carruthers V B, Moreno S N J, Sibley L D. 1999. Ethanol and acetaldehyde elevate intracellular [Ca2+] calcium and stimulate microneme discharge in Toxoplasma gondii. Biochem J 342:379-386.
4. Carruthers V B, Sibley L D. 1999. Mobilization of intracellular calcium stimulates microneme discharge in Toxoplasma gondii. Mol Microbiol 31:421-428.
5. Huynh M H, Barenau K E, Harper J M, Beatty W L, Sibley L D, Carruthers V B. 2003 Rapid invasion of host cells by Toxoplasma requires secretion of the MIC2-M2AP adhesive protein complex. EMBO J 22:2082-2090.
6. Brydges S D, Sherman G D, Nockemann S, Loyens A, Daubener W, Dubremetz J, Carruthers V B. 2000. Molecular characterization of TgMIC5, a proteolytically processed antigen secreted from the micronemes of Toxoplasma gondii. Mol Biochem Parasitol 111:51-66.
7. Hoff E F, Cook S H, Sherman G D, Harper J M, Ferguson D J, Dubremetz J F, Carruthers V B. 2001. Toxoplasma gondii: molecular cloning and characterization of a novel 18-kDa secretory antigen, TgMIC10. Exp Parasitol 97:77-88.
8. Mercier C, Cesbron-Delauw M F. 2015. Toxoplasma secretory granules: one population or more? Trends Parasitol 31:60-71.
9. Black C A. 1999. Delayed type hypersensitivity: current theories with an historic perspective. Dermatol Online J 5:7.
10. Allen I C. 2013. Delayed-type hypersensitivity models in mice. Methods Mol Biol 1031:101-107.
11. Rougier D, Ambroise-Thomas P. 1985. Detection of toxoplasmic immunity by multipuncture skin test with excretory-secretory antigen. Lancet 2:121-123.
12. Veprekova. 1978. Approximative molecular weight of the active component in toxoplasmin. Folia Parasitol (Praha) 25:273-275.
13. Frenkel J K. 1948. Dermal hypersensitivity to toxoplasma antigens (toxoplasmins). Proc Soc Exp Biol Med 68:634-639.
14. Gross S, Gammon S T, Moss B L, Rauch D, Harding J, Heinecke J W, Ratner L, Piwnica-Worms D. 2009. Bioluminescence imaging of myeloperoxidase activity in vivo. Nat Med 15:455-461

### REFERENCES FOR EXAMPLES 5-6

1 Carruthers V B, Sibley L D. 1997. Sequential protein secretion from three distinct organelles of Toxoplasma gondii accompanies invasion of human fibroblasts. Eur J Cell Biol 73:114-123.
2. Carruthers V B, Giddings O K, Sibley L D. 1999. Secretion of micronemal proteins is associated with Toxoplasma invasion of host cells. Cell Microbiol 1:225-236.
3. Carruthers V B, Moreno S N J, Sibley L D. 1999. Ethanol and acetaldehyde elevate intracellular [Ca2+] calcium and stimulate microneme discharge in Toxoplasma gondii. Biochem J 342:379-386.
4. Carruthers V B, Sibley L D. 1999. Mobilization of intracellular calcium stimulates microneme discharge in Toxoplasma gondii. Mol Microbiol 31:421-428.
5. Huynh M H, Barenau K E, Harper J M, Beatty W L, Sibley L D, Carruthers V B. 2003. Rapid invasion of host cells by Toxoplasma requires secretion of the MIC2-M2AP adhesive protein complex. EMBO J 22:2082-2090.
6. Brydges S D, Sherman G D, Nockemann S, Loyens A, Daubener W, Dubremetz J, Carruthers V B. 2000. Molecular characterization of TgMIC5, a proteolytically processed antigen secreted from the micronemes of Toxoplasma gondii. Mol Biochem Parasitol 111:51-66.
7 Hoff E F, Cook S H, Sherman G D, Harper J M, Ferguson D J, Dubremetz J F, Carruthers V B. 2001. Toxoplasma gondii: molecular cloning and characterization of a novel 18-kDa secretory antigen, TgMIClO. Exp Parasitol 97:77-88.
8. Mercier C, Cesbron-Delauw M F. 2015. Toxoplasma secretory granules: one population or more? Trends Parasitol 31:60-71.
9. Black C A. 1999. Delayed type hypersensitivity: current theories with an historic perspective. Dermatol Online J 5:7.
10. Allen I C. 2013. Delayed-type hypersensitivity models in mice. Methods Mol Biol 1031:101-107.
11. Rougier D, Ambroise-Thomas P. 1985. Detection of toxoplasmic immunity by multipuncture skin test with excretory-secretory antigen. Lancet 2:121-123.
12. Veprekova. 1978. Approximative molecular weight of the active component in toxoplasmin. Folia Parasitol (Praha) 25:273-275.
13. Frenkel J K. 1948. Dermal hypersensitivity to toxoplasma antigens (toxoplasmas]. Proc Soc Exp Biol Med 68:634-639.
14. Gross S, Gammon S T, Moss B L, Rauch D, Harding J, Heinecke J W, Ratner L, Piwnica-Worms D. 2009. Bioluminescence imaging of myeloperoxidase activity in vivo. Nat Med 15:455-461.

### REFERENCES FOR EXAMPLES 7

1 Huynh M H, Barenau K E, Harper J M, Beatty W L, Sibley L D, Carruthers V B. 2003. Rapid invasion of host cells by Toxoplasma requires secretion of the MIC2-M2AP adhesive protein complex. EMBO J 22:2082-2090.
2. Brydges S D, Sherman G D, Nockemann S, Loyens A, Daubener W, Dubremetz J, Carruthers V B. 2000. Molecular characterization of TgMIC5, a proteolytically processed antigen secreted from the micronemes of Toxoplasma gondii. Mol Biochem Parasitol 111:51-66.
3. Hoff E F, Cook S H, Sherman G D, Harper J M, Ferguson D J, Dubremetz J F, Carruthers V B. 2001. Toxoplasma gondii: molecular cloning and characterization of a novel 18-kDa secretory antigen, TgMIClO. Exp Parasitol 97:77-88.
4. Mercier C, Cesbron-Delauw M F. 2015. Toxoplasma secretory granules: one population or more? Trends Parasitol 31:60-71.
5. Black C A. 1999. Delayed type hypersensitivity: current theories with an historic perspective. Dermatol Online J 5:7.
6. Allen I C. 2013. Delayed-type hypersensitivity models in mice. Methods Mol Biol 1031:101-107.
7 Rougier D, Ambroise-Thomas P. 1985. Detection of toxoplasmic immunity by multipuncture skin test with excretory-secretory antigen. Lancet 2:121-123.
8. Veprekova. 1978. Approximative molecular weight of the active component in toxoplasmin. Folia Parasitol (Praha) 25:273-275.
9. Frenkel J K. 1948. Dermal hypersensitivity to toxoplasma antigens (toxoplasmins). Proc Soc Exp Biol Med 68:634-639.
10. Gross S, Gammon S T, Moss B L, Rauch D, Harding J, Heinecke J W, Ratner L, Piwnica-Worms D. 2009. Bioluminescence imaging of myeloperoxidase activity in vivo. Nat Med 15:455-461.
11. Philpott D J, Girardin S E. 2004. The role of Toll-like receptors and Nod proteins in bacterial infection. Molec Immunol 41:1099-1108.
12. Jacobs D M, Morrison D C. 1977. Inhibition of the mitogenic response to lipopolysaccharide (LPS) in mouse spleen cells by polymyxin B. J Immunol 118:2127.
13. Nielsen M, Lund O, Buus S, Lundegaard C. 2010. WIC class II epitope predictive algorithms. Immunology 130:319-328.
14. Wang P, Sidney J, Dow C, Mothe B, Sette A, Peters B. 2008. A systematic assessment of MHC class II peptide binding predictions and evaluation of a consensus approach. PLoS Comput Biol 4:e1000048.
15. Erskine C L, Krco C), Hedin K E, Borson N D, Kalli K R, Behrens M D, Heman-Ackah S M, von Hofe E, Wettstein P J, Mohamadzadeh M, Knutson K L. 2011. MHC class II epitope nesting modulates dendritic cell function and improves generation of antigen-specific CD4 helper T cells. J Immunol 187:316-324.

1. An in vitro method of monitoring a T cell response in a T cell from a subject, wherein the subject has not been immunized against Toxoplasma gondii, the method comprising:
contacting the T cell of the subject with a composition comprising an isolated Toxoplasma gondii antigen selected from the group consisting of:
a. isolated and purified MIC4;
b. truncated MIC4;
c. extended MIC4; and
d. combinations thereof, and
detecting or quantifying release of a cytokine.

2. The method of claim 1 wherein the response is a delayed-type hypersensitivity response.

3. The method of claim 1 wherein the response is release of a cytokine.

4. The method of claim 1 wherein the response is release of interferon-γ.

5. The method of claim 1 wherein the T cells are in peripheral blood mononuclear cells.

6. The method of claim 1 wherein the T cells are in a blood sample.

7. The method of claim 1 further comprising the step of detecting or quantifying release of interferon-γ.


SEQUENCE LISTING (64 sequences)

SEQ ID NO: 1 - protein, 456 aa, Toxoplasma gondii
MGQALFLTVLLPVLFGVGPEAYGEASHSHSPASGRYIQQMLDQRCQEIAAELCQGGLRKMCVPSSRIVARNAVGITHQNTLEWRCFDTASLLESNQENNGVNCVDDCGHTIPCPGGVHRQNSNHATRHEILSKLVEEGVQRFCSPYQASANKYCNDKFPGTIARRSKGFGNNVEVAWRCYEKASLLYSVYAECASNCGTTWYCPGGRRGTSTELDKRHYTEEEGIRQAIGSVDSPCSEVEVCLPKDENPPVCLDESGQISRTGGGPPSQPPEMQQPADRSDERGGGKEQSPGGEAQPDHPTKGGNIDLPEKSTSPEKTPKTEIHGDSTKATLEEGQQLTLTFISTKLDVAVGSCHSLVANFLDGFLKFQTGSNSAFDVVEVEEPAGPAVLTIGLGHKGRLAVVLDYTRLNAALGSAAYVVEDSGCSSSEEVSFQGVGSGATLVVTTLGESPTAVSA

SEQ ID NO: 2 - protein, 321 aa, Toxoplasma gondii
MAYGEASHSHSPASGRYIQQMLDQRCQEIAAELCQGGLRKMCVPSSRIVARNAVGITHQNTLEWRCFDTASLLESNQENNGVNCVDDCGHTIPCPGGVHRQNSNHATRHEILSKLVEEGVQRFCSPYQASANKYCNDKFPGTIARRSKGFGNNVEVAWRCYEKASLLYSVYAECASNCGTTWYCPGGRRGTSTELDKRHYTEEEGIRQAIGSVDSPCSEVEVCLPKDENPPVCLDESGQISRTGGGPPSQPPEMQQPADRSDERGGGKEQSPGGEAQPDHPTKGGNIDLPEKSTSPEKTPKTEIHGDSTKATLEEGQQLTL

SEQ ID NO: 3 - DNA, 1920 bp, Toxoplasma gondii
  source 1..1920: mol_type = other DNA
acctgaaagcgggtgccgcgtcgctaccgtttcctgtggcgtctctagtgcgacatccgaagtaacagtaacgtccggcatggaacgccgacgcgggtgttccagtcgcctggctccttctactcgcacttcgatgttacgttccttattggtgcgacgcggttctcgtgttgctagacgtcgcaccggctgaaagctgtagaaaatttagttattttcctgtcagctagcttgcaggagtgcgtttttgtgtgttggtttcgtctcacatggctgctgatctgttgatgcagctgtgtacacgtgcctcgattctgtagttgacctagaacggatttgcaaagatgggccaggcgttgtttctcaccgttctattgccggtgttatttggcgttgggccagaagcatatggagaagcgtcgcattctcattcgccggcatcgggacgttatatacaacagatgcttgaccaacgctgccaagagattgctgcagaactctgccaaggcggacttcgtaaaatgtgtgtgccctctagccggatagtagctcgaaacgccgtgggcattactcatcaaaatacacttgaatggagatgctttgatacagcctctttgctggagagcaatcaagaaaacaacggtgttaattgcgtggacgactgtggccacacgataccgtgtcctggcggcgtacaccggcaaaacagtaatcacgcaacgcgccatgagatactgtccaaattggtcgaagaaggagtacaacggttctgcagtccttatcaagcatctgccaacaagtactgtaacgacaaatttccagggaccattgcgaggaggtcgaagggcttcggaaacaatgtcgaggttgcgtggaggtgttacgagaaggccagcttgctgtactcggtttatgctgagtgtgcgagcaactgcggaacaacgtggtactgccctggaggacgacgagggacgtcgacagaactagacaagcggcattatacagaagaggaaggaattcgccaggcaatcggatccgtcgacagcccatgttctgaagttgaagtctgcctaccgaaggatgagaatcccccggtgtgtttagatgaaagtggccagatttcacgaactggtggtgggccaccgtcacaaccgcctgagatgcaacagcccgccgatcgttcggacgagagaggtggcggtaaggaacagtcgcctggaggagaagctcagccggaccatccaacgaagggtggtaacatagacctgcctgagaaatcaacatctcccgagaagacgccgaaaaccgagatccatggtgacagcacgaaagcgacgctcgaagaggggcagcaactaacgctcacgtttatctccactaaactggatgttgctgtaggctcgtgtcattcactcgtcgcgaatttccttgatggatttttgaagtttcagacgggctcaaattcggcgttcgatgtggtagaagtggaagagccagcaggacccgcagtgcttacgataggtctgggacacaaaggccgtctcgctgttgtcctcgactacaccaggctcaatgctgctttaggatcagctgcttacgtggtcgaagattctggatgcagctcaagtgaagaggttagtttccaaggagtgggtagtggagcgacgctcgtggtgacgacgcttggcgagagtcctacggccgtctctgcttgatttatagtactctttggagcatgcttgtggaggaacgggacaatctcggcaaaatcaggatgaagtttgtgagatacagatcgttcctgaacagtggaagatgcgtcactattacacctatatgcgtcctggttcttgtagagttggagttcttgcaggtgtaatgactatgacatacggatataacttcatacggggaactgtg

SEQ ID NO: 4 - DNA, 48 bp, Toxoplasma gondii
  source 1..48: mol_type = unassigned DNA
actgtggtctctaggtatggaagcatatggagaagcgtcgcattctca

SEQ ID NO: 5 - DNA, 49 bp, Toxoplasma gondii
  source 1..49: mol_type = unassigned DNA
actgttctagatcagagcgttagttgctgcccctcttcgagcgtcgctt

SEQ ID NO: 6 - protein, 198 aa, Toxoplasma gondii
MALSSLNNIRPFSGLLGCGLLFGALVVVVACVFSVPVEAGVLRKVAGAGSLQASIGEHDFFNDYDQDEEYRKRQQELQNQSPEEVEEAKRKYHEELRRKAEEDAETKRKQEAVIQELKEVAKKRGLREAAEREEKRIDEQQANYEQRQQELRDMDSAMEERLMQQRKKDQEERELARKNSDKVMEELKEKLARRRKSM

SEQ ID NO: 7 - protein, 198 aa, Toxoplasma gondii; identical to SEQ ID NO: 6

SEQ ID NO: 8 - DNA, 960 bp, Toxoplasma gondii
  source 1..960: mol_type = unassigned DNA
ttgtcggatagaacacatcaaaaattttttctgtttcatctgtcacgtgcgtccttttggtggaagcgacgcgtcgtcgcggccctgttgggggtagtcaatatttcagtaagcgtttctagcgacacccgctacacagaacactcggttgaagcactgtgatgctggcgtttctgtccgctgtctttctgcagaatggcgctttcttctttgaacaatatcaggccttttagcgggttgctgggttgcggcctgctgtttggcgcccttgtggtcgtggtggcatgtgttttcagcgttcctgtggaagcaggcgttcttcgtaaggtagcaggtgcaggaagtctccaggcgtctattggagagcacgatttttttaacgattacgatcaggacgaggaatacaggaagcgccagcaagaactgcagaatcagagtccagaagaagtcgaggaagcgaaacgcaaataccacgaagagctgagacggaaagcagaagaagatgcagagacgaaacgtaagcaagaagcagtcattcaagaactgaaagaggtggcaaagaaaagaggacttcgtgaagccgctgagcgtgaggagaagcgcattgatgagcagcaggctaattacgagcaacgacaacaagaactgagagacatggattcagcaatggaggagaggcttatgcagcagagaaaaaaagaccaggaagagagagaacttgcaagaaaaaacagcgataaggtcatggaggagctcaaagagaaactcgcaagacgcaggaaatcaatgtagaagaaaggcaagttctgactagtgtatgaagtgtagcagcggtccgttattaccgtgtatacagggagatcgtcgttcagtttgaagtgtttattccgtgttgaaagaagcgtttgtgtaatgcagatgatcctgagatgatagtgcgggaaatcgaatgactcattg

SEQ ID NO: 9 - DNA, 41 bp, Toxoplasma gondii
  source 1..41: mol_type = unassigned DNA
actgtggtctctaggtatggcgctttcttctttgaacaata

SEQ ID NO: 10 - DNA, 38 bp, Toxoplasma gondii
  source 1..38: mol_type = unassigned DNA
actgttctagatcacattgatttcctgcgtcttgcgag

SEQ ID NO: 11 - protein, 236 aa, Toxoplasma gondii
MARHAIFFALCVLGLVAAALPQFATAATASDDELMSRIRNSDFFDGQAPVDSLRPTNAGVDSKGTDDHLTTSMDKASVESQLPRREPLETEPDEQEEVHFRKRGVRSDAEVTDDNIYEEHTDRKVVPRKSEGKRSFKDLLKKLALPAVGMGASYFAADRILPELTEQQQTGEEPLTTGQNVSTVLGFAALAAAAAFLGMGLTRTYRHFSPRKNRSRQPALEQEVPESGKDGEDARQ

SEQ ID NO: 12 - protein, 236 aa, Toxoplasma gondii; identical to SEQ ID NO: 11

SEQ ID NO: 13 - DNA, 1120 bp, Toxoplasma gondii
  source 1..1120: mol_type = unassigned DNA
ttactttcttcggattacattcttccactaaaagctggttttgtccagtatccattcgtcgctaccgttgcgcagtcacgttgaattttgcagcggcaaaacatcttgtgtaaaattcgagttttgttgatgattgaagtaccctgtattggggcttgctaacgttttgtattaaaagggtttactgcggcgtctcatttccaaaatggcccgacacgcaatttttttcgcgctttgtgttttaggcctggtggcggcggctttgccccagttcgctaccgcggccaccgcgtcagatgacgaactgatgagtcgaatccgaaattctgactttttcgatggtcaagcacccgttgacagtctcagaccgacgaacgccggtgtcgactcgaaagggaccgacgatcacctcaccaccagcatggataaggcatctgtagagagtcagcttccgagaagagagccattggagacggagccagatgaacaagaagaagttcatttcaggaagcgaggcgtccgttccgacgctgaagtgactgacgacaacatctacgaggagcacactgatcgtaaagtggttccgaggaagtcggagggcaagcgaagcttcaaagacttgctgaagaagctcgcgctgccggctgttggtatgggtgcatcgtattttgccgctgatagaattctgccggaactaacagagcagcaacagacaggcgaagaacccctaaccaccggccagaatgtgagcactgtgttaggcttcgcagcgcttgctgctgccgcagcgttccttggcatgggtctcacgaggacgtaccgacatttttccccacgcaaaaacagatcacggcagcctgcactcgagcaagaggtgcctgaatcaggcaaagatggggaggatgcccgccagtaggatatgggggctaataaaagtgagtaggagctcgaggacagtgtcccgaacgcgcctgagaggcagacagacacagaagagtgaagaaaaacaacatggtattacgtgcggtgagtgtttgctgtcacgtgttttttgcgccacaaagacagcttgtgttgtatgcatgggatcgacagttcatggacggcgctacccagagag

SEQ ID NO: 14 - DNA, 37 bp, Toxoplasma gondii
  source 1..37: mol_type = unassigned DNA
actgtgaggactcaggtatggctttgccccagttcgc

SEQ ID NO: 15 - DNA, 35 bp, Toxoplasma gondii
  source 1..35: mol_type = unassigned DNA
actgttctagatcactggcgggcatcctccccatc

SEQ ID NO: 16 - DNA, 324 bp, Toxoplasma gondii
  source 1..324: mol_type = unassigned DNA
atgggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcaggcccctgaagatttggacatggaggataacgatattattgaggctcaccgcgaacagattggaggt

SEQ ID NO: 17 - protein, 108 aa, Toxoplasma gondii
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGG

SEQ ID NO: 18 - protein, 456 aa, Toxoplasma gondii; identical to SEQ ID NO: 1

SEQ ID NO: 19 - protein, 456 aa, Toxoplasma gondii; identical to SEQ ID NO: 1

SEQ ID NO: 20 - protein, 456 aa, Toxoplasma gondii; identical to SEQ ID NO: 1

SEQ ID NO: 21 - protein, 456 aa, Toxoplasma gondii; identical to SEQ ID NO: 1

SEQ ID NO: 22 - protein, 456 aa, Toxoplasma gondii
MGQALFLTVLLPVLFGVGPEAYGEASHSHSPASGRYIQQMLDQRCQEIAAELCQSGLRKMCVPSSRIVARNAVGITHQNTLQWRCFDTASLLESNQENNGVNCVDDCGHTIPCPGGVHRQNSNHATRHEILSKLVEEGVQRFCSPYQASANKYCNDKFPGTIARRSKGFGNNVEVAWRCYEKASLLYSVYAECASNCGTTWYCPGGRRGTSTELDKRHYTEEEGIRQAIGSVDSPCSEVEVCLPKDENPPVCLDESGQISRTGGGPPSQPPEMQQPADRSDERGGGKEQSPGGEAQPDHPTKGGNIDLPEKSTSPEKTPKTEIHGDSTKATLEEGQQLTLTFISTKLDVAVGSCHSLVANFLDGFLKFQTGSNSAFDVVEVEEPAGPAVLTIGLGHKGRLAVVLDYTRLNAALGSAAYVVEDSGCSSSEEVSFQGVGSGATLVVTTLGESPTAVSA

SEQ ID NO: 23 - protein, 456 aa, Toxoplasma gondii
MGQALFLTVLLPVLFGVGPEAYGEASHSHSPASGRYIQQMLDQRCQEIAAELCQSGLRKMCVPSSRIVARNAVGITHQNTLQWRCFDTASLLESNQENNGVNCVDDCGHTIPCPGGVHRQNSNHATRHEILSKLVEEGVQRFCSPYQASANKYCNDKFPGTIARRSKGFGNNVEVAWRCYEKASLLYSVYAECASNCGTTWYCPGGRRGTSTELDKRHYTEEEGIRQAIGSVDSPCSEVEVCLPKDENPPLCLDESGQISRTGGGPPSQPPEMQQPADRSDERGGGKEQSPGGEAQPDHPTKGGNIDLPEKSTSPEKTPKTEIHGDSTKATLEEGQQLTLTFISTKLDVAVGSCHSLVANFLDGFLKFQTGSNSAFDVVEVEEPAGPAVLTIGLGHKGRLAVVLDYTRLNAALGSAAYVVEDSGCSSSEEVSFQGVGSGATLVVTTLGESPTAVSA

SEQ ID NO: 24 - protein, 456 aa, Toxoplasma gondii; identical to SEQ ID NO: 23

SEQ ID NO: 25 - protein, 456 aa, Toxoplasma gondii; identical to SEQ ID NO: 23

SEQ ID NO: 26 - protein, 456 aa, Toxoplasma gondii; identical to SEQ ID NO: 23

SEQ ID NO: 27 - protein, 456 aa, Toxoplasma gondii
MGQALFLTVLLPVLFGVGPEAYGEASHSHSPASGRYIQQMLDQRCQEIAAELCQSGLRKMCVPSSRIVARNAVGITHQNTLQWRCFDTASLLESNQENNGVNCVDDCGHTIPCPGGVHRQNSNHATRHQILSKLVEEGVQRFCSPYQASANKYCNDKFPGTIARRSKGFGNNVEVAWRCYEKASLLYSVYAECASNCGTTWYCPGGRRGTSTELDKRHYTEEEGIRQAIGSVDSPCSEVEVCLPKDENPPLCLDESGQISRTGGGPPSQPPEMQQPADRSDERGGGKEQSPGGEAQPDHPTKGGNIDLPEKSTSPEKTPKTEIHGDSTKATLEEGQQLTLTFISTKLDVAVGSCHSLVANFLDGFLKFQTGSNSAFDVVEVEEPAGPAVLTIGLGHKGRLAVVLDYTRLNAALGSAAYVVEDSGCSSSEEVSFQGVGSGATLVVTTLGESPTAVSA

SEQ ID NO: 28 - protein, 456 aa, Toxoplasma gondii
MGQALFLTVLLPVLFGVGPEAYGEASHSHSPASGRYIQQMLDQRCQEIAAELCQSGLRKMCVPSSRIVARNAVGITHQNTLQWRCFDTASLLESNQENNGVNCVDDCGHTIPCPGGVHRQNSNHATRHEILSKLVEEGVQRFCSPYQASANKYCNDKFPGTIARRSKGFGNNVEVAWRCYEKASLLYSVYAECASNCGTTWYCPGGRRGTSTELDKRHYTEEEGIRQAIGSVDSPCSEVEVCLPKDENPPLCLDESGQISRTGGGPPSQPPEMQQPADRSDERGGGKEQSPGGEAQPDHPTKGGNIDLPEKSTSPEKTPKTEIHGDSTKATLEEGQQLTLTFISTKLDVAVGSCHSLVANFLDGFLKFQTGSNSAFDVVEVEDPAGPAVLTIGLGHKGRLAVVLDYTRLNAALGSAAYVVEDSGCSSSEEVSFQGVGSGATLVVTTLGESPTAVSA

SEQ ID NO: 29 - protein, 456 aa, Toxoplasma gondii; identical to SEQ ID NO: 22

SEQ ID NO: 30 - protein, 456 aa, Toxoplasma gondii; identical to SEQ ID NO: 22

SEQ ID NO: 31 - protein, 456 aa, Toxoplasma gondii; identical to SEQ ID NO: 1

SEQ ID NO: 32 - protein, 456 aa, Toxoplasma gondii; identical to SEQ ID NO: 23

SEQ ID NO: 33 - protein, 456 aa, Toxoplasma gondii; identical to SEQ ID NO: 23

SEQ ID NO: 34 - protein, 198 aa, Toxoplasma gondii; identical to SEQ ID NO: 6

SEQ ID NO: 35 - protein, 198 aa, Toxoplasma gondii; identical to SEQ ID NO: 6

SEQ ID NO: 36 - protein, 198 aa, Toxoplasma gondii; identical to SEQ ID NO: 6

SEQ ID NO: 37 - protein, 198 aa, Toxoplasma gondii; identical to SEQ ID NO: 6

SEQ ID NO: 38 - protein, 198 aa, Toxoplasma gondii; identical to SEQ ID NO: 6

SEQ ID NO: 39 - protein, 198 aa, Toxoplasma gondii; identical to SEQ ID NO: 6

SEQ ID NO: 40 - protein, 198 aa, Toxoplasma gondii; identical to SEQ ID NO: 6

SEQ ID NO: 41 - protein, 198 aa, Toxoplasma gondii; identical to SEQ ID NO: 6

SEQ ID NO: 42 - protein, 198 aa, Toxoplasma gondii
MALSSLNNIRPFSGLLGCGLLFGALVVVVACVFSVPVEAGVLRKVAGAGSLQASIGEHDFFNDYDQDEEYRKRQQELQNQSPEEVEEAKRKYHEELRRKAEEDAETKRKQEAVIQELKEVAKKRGLREAAEREEKRTDEQQANYEQRQQELRDMDSAMEERLMQQRKKDQEERELARKNSDKVMEELKEKLARRRKSM

SEQ ID NO: 43 - protein, 198 aa, Toxoplasma gondii
MALSSLNNIRPFSGLLGCGLLFGALVVVVACAFSVPVEAGVLRKVAGAGSLQASIGEHDFFNDYDQDEEYRKRQQELQNQSPEEVEEAKRKYHEELRRKAEEDAETKRKQEAVIQELKEVAKKRGLREAAEREEKRTDEQQANYEQRQQELRDMDSAMEERLMQQRKKDQEERELARKNSDKVMEELKEKLARRRKSM

SEQ ID NO: 44 - protein, 167 aa, Toxoplasma gondii
MLVFLSAVFLQNGAFFFEQYQASIGEHDFFNDYDQDEEYRKRQQELQNQSPEEVEEAKRKYHEELRRKAEEDAETKRKQEAVIQELKEVAKKRGLREAAEREEKRIDEQQANYEQRQQELRDMDSAMEERLMQQRKKDQEERELARKNSDKVMEELKEKLARRRKSM

SEQ ID NO: 45 - protein, 198 aa, Toxoplasma gondii; identical to SEQ ID NO: 6

SEQ ID NO: 46 - protein, 198 aa, Toxoplasma gondii; identical to SEQ ID NO: 6

SEQ ID NO: 47 - protein, 198 aa, Toxoplasma gondii; identical to SEQ ID NO: 6

SEQ ID NO: 48 - protein, 198 aa, Toxoplasma gondii; identical to SEQ ID NO: 6

SEQ ID NO: 49 - protein, 198 aa, Toxoplasma gondii; identical to SEQ ID NO: 6

SEQ ID NO: 50 - protein, 383 aa, Toxoplasma gondii
MRGGTSALLHALTFSGAVWMCTPAEALPIQKSVQLGSFDKVVPSREVVSESLAPSFAVTETHSSVQSPSKQETQLCAISSEGKPCRNRQLHTDNGYFIGASCPKSACCSKTMCGPGGCGEFCSSNWIFCSSSLIYHPDKSYGGDCSCEKQGHRCDKNAECVENLDAGGGVHCKCKDGFVGTGLTCSEDPCSKRGNAKCGPNGTCIVVDSVSYTCTCGDGETLVNLPEGGQGCKRTGCHAFRENCSPGRCIDDASHENGYTCECPTGYSREVTSKAEESCVEGVEVTLAEKCEKEFGISASSCKCDNGYSGSASATSHHGKGESGSEGSLSEKMNIVFKCPSGYHPRYHAHTVTCEKIKHFALDGAGNHDTTTYVARRRYPASL

SEQ ID NO: 51 - protein, 249 aa, Toxoplasma gondii
YHPDKSYGGDCSCEKQGHRCDKNAECVENLDAGGGVHCKCKDGFVGTGLTCSEDPCSKRGNAKCGPNGTCIVVDSVSYTCTCGDGETLVNLPEGGQGCKRTGCHAFRENCSPGRCIDDASHENGYTCECPTGYSREVTSKAEESCVEGVEVTLAEKCEKEFGISASSCKCDNGYSGSASATSHHGKGESGSEGSLSEKMNIVFKCPSGYHPRYHAHTVTCEKIKHFALDGAGNHDTTTYVARRRYPASL

SEQ ID NO: 52 - DNA, 3837 bp, Toxoplasma gondii
  source 1..3837: mol_type = unassigned DNA
tcttctcttcttccgtacttttccctgcatttcacacccctggtatgactccacaccgcgtgtaaatgtcccttaggtgacacccgcagcagcgcgtaggaggaagtagatgtcagtgtagacgtttttgagatgagagacgataacgtaaaatgccgccgataacttctgcattatacacactctctctccacgcctaggatgacaggtacggcggcacacggaggaaagtgggggggggggggggggcgaacagaaaggtcacatggaaggccgctcgactctccactcacgaagtgaaggcttcgtcccgttttgctggacaacgaatgcgaacttcttcactcgcttgtgacacacacaactccagaggcacagagatgtgaagcagaagagtggcgtgtgcgtcgcttctgtcggcggcaagccccgctccgtctctttggtggcgattctggtgtgcaccgtgtgccaagaagttgcgtgtcacgcgacttttggaaatgcatcaggttcagagtcgttatgttgcgattcaggctctcggcagagaatcatttccctgtaagctagttgaactcgcctttttaaaagcggcagcagtgcccttgtggaaggcctcactgtgcctactttcctcgtcctgagtttttccgccttcggcctcattttttgctcaccaaaatcgtgtcctaccgtcaagttttgccatagactcctacgggaaaaaacaagccggtcgacacggacgacgcccgcagggaagcgtcccctccgcagaaatcgggagacaactgtcgttgacggtgctgcgcgaaaggtcacagagtttccagtgtgttcatcagacctcactgtgcactgttagcggccgctgtcccgcctggtcaacaagtatcacaccctcgtccccgccattggcacggagctcgatgagctgcagtgtcgcttttaggggagtcgtgcaatcacgccgcaacacaggcgtgattcgatcttcaattgctaggtaaccactcgtgcttggtagctctgcaatggctcgagcgacgggggtgatgcaacatgctgctaaaaactcgacagacgtgtcaccggaacccacctaaataggagaccacgggtctctggtgtgtcgcgtcgcattctcgcgtcgcattctcgcgtcgcaatgaccggccagttgctcgacgtcgccagccgggactgaagagcgttcatcgagtcagcagcattgcgtccccttgctcggtgaaaaaagactctctggtcgagtctagctcgtgtcacttctgtttctaacctccttcgttcaccggtacacctccgatgtgacttttggtacacttgccctgtcgcacgacgcacgctgtcactcaacttgctgctagcgcaatcgataggttccctcgaaccagccatcacacacacaccttttccgggaagacgtttgcgggcggtgggtcgcagctcgtcgagagtgcgtttctgtgcatttctgtgggcagtgcagcgcgtttgcgcgccttactctgtgtgtaacttccttgtccaacactggtaaaaatgcgaggcgggacgtccgcgctgttgcacgcgctcaccttcagtggggccgtgtggatgtgcaccccagcggaggctttgccgattcagaagtctgtgcagctgggcagctttgacaaagttgtgccgagccgcgaagtcgtctctgagagtcttgctccgtctttcgcggtgactgagactcactcgtctgtgcaatcccccagcaagcaggagacgcaactctgtgctatctcgagtgaaggcaagccatgtcgaaaccgtcagttgcacactgacaacgggtacttcatcggggccagttgccccaagagcgcttgctgcagcaagaccatgtgcggccccggcggctgcggagaattctgctccagcaactggattttttgcagcagttcgctcatctaccatcctgacaaaagctatggaggagactgcagctgtgaaaagcagggccatcggtgcgacaaaaacgcagaatgcgtcgaaaacttggacgcgggtgggggtgtgcactgcaagtgcaaagacggcttcgtcggcactgggttgacttgctccgaggatccttgttcaaaaagagggaacgcgaagtgcggacccaacgggacgtgcatcgtcgtcgattcagtcagctacacatgcacctgcggcgacggcgaaactctagtgaacctcccggaagggggacaaggatgcaagaggactggatgtcatgccttcagggagaactgcagccctggtagatgtattgatgacgcctcgcatgagaatggctacacctgcgagtgccccacagggtactcacgtgaggtgacttccaaggcggaggagtcgtgtgtggaaggagtcgaagtcacgctggctgagaaatgcgagaaggaattcggcatcagcgcgtcatcctgcaaatgcgataacggatactccggatctgcttccgcaacctcccaccatgggaaaggagaatcgggatccgaggggagcttgagtgaaaaaatgaatattgtcttcaagtgccccagtggctaccatccaagataccatgcccacaccgtgacgtgtgagaaaattaagcactttgcccttgacggggccggcaaccacgacacgactacgtatgtcgcaagacgaaggtacccagcgagtctctgagagcggagatcagcgcaaagacaagatgcagagtttgactcgagaaacaatagtaacacgaagtaaaaagtctccacactaagccaaggattgagaatatttcgatttgtgccgctggcaatagtggccttggcctagaaagaagttctgcaacgaagcgatcggctcacacgcggatacacagatgggtttgtaccgagaacgttaggtttgtgaaccgagttcaggtaaaacaaagtagattgtgcctttacgcagacagcgagggaaaacatgaggacacactgccaactaaagcaagactgcctcactaattaccaccgacacacgacatggttacccccgcgttttgccgcgtgcaaagtttgaattctgatggttctcgagtctgaaagcctaaaccgcccaaccatgtatgaaataagaacccatcaaacgtgagacatctctgccgaagtgcctacgaaaagaacgcttctgccactaggaggtgcggcctcttcattctatgagaacctgctttgtcggtgtcaacctctggggaaatcgcctgcctttacacattttgctcgttgtagagcaagggatctgttgctgcgtttactccaatacaatgatcgccgtttcgctgtaggcaagcgatccgaaaatgtacgttcgagtcagcagctacttgagaagcagccaacgccgacacttgctgcgtttgactgaggtgcactcgcaaacagtctcgtctccccggggcaatttctgagagaaatgcgggaatggacgtaatggtgctcttctgtgagtgctcttccaccaatttttcgacaagtgttttcgtgacagtcgagtataccttcttatgtcattctgtctccgtcagtgctatcggattcttcctattcctctaccctttctacagtcgcatacaaagctgctgaaacaagacttcctttgtctagggtagttgtacactccacacatatctgactgaaacctacggcaggaagtctggtcggcactgtgcttccttgttggcttttcgtcgtttctttgtctacgagcttcactgggtccttgacacggcttgtgagcgttgtgctcaatattcgaccagctgtatttgtg

SEQ ID NO: 53 - DNA, 53 bp, Toxoplasma gondii
  source 1..53: mol_type = unassigned DNA
actgtggtctctaggtatgatctaccatcctgacaaaagctatggaggagact

SEQ ID NO: 54 - DNA, 39 bp, Toxoplasma gondii
  source 1..39: mol_type = unassigned DNA
acttgttctagatcagagactcgctgggtaccttcgtct

SEQ ID NO: 55 - protein, 580 aa, Toxoplasma gondii
MRASLPVHLVVCTQLSAVWFGVAKAHGGHRLEPHVPGFLQGFTDITPAGDDVSANVTSSEPAKLDLSCVHSDNKGSRAPTIGEPVPDVSLEQCAAQCKAVDGCTHFTYNDDSKMCHVKEGKPDLYDLTGGKTASRSCDRSCFEQHVSYEGAPDVMTAMVTSQSADCQAACAADPSCEIFTYNEHDQKCTFKGRGFSAFKERGVLGVTSGPKQFCDEGGKLTQEEMEDQISGCIQLSDVGSMTADLEEPMEADSVGACMERCRCDGRCTHFTFNDNTRMCYLKGDKMQLYSSPGDRTGPKSCDSSCFSNGVSYVDDPATDVETVFEISHPIYCQVICAANPLCTVFQWYASEAKCVVKRKGFYKHRKTGVTGVTVGPREFCDFGGSIRDREEADAVGSDDGLNAEATMANSPDFHDEVECVHTGNIGSKAQTIGEVKRASSLSECRARCQAEKECSHYTYNVKSGLCYPKRGKPQFYKYLGDMTGSRTCDTSCLRRGVDYSQGPEVGKPWYSTLPTDCQVACDAEDACLVFTWDSATSRCYLIGSGFSAHRRNDVDGVVSGPYTFCDNGENLQVLEAKDTE

SEQ ID NO: 56 - protein, 173 aa, Toxoplasma gondii
SEPAKLDLSCVHSDNKGSRAPTIGEPVPDVSLEQCAAQCKAVDGCTHFTYNDDSKMCHVKEGKPDLYDLTGGKTASRSCDRSCFEQHVSYEGAPDVMTAMVTSQSADCQAACAADPSCEIFTYNEHDQKCTFKGRGFSAFKERGVLGVTSGPKQFCDEGGKLTQEEMEDQISG

SEQ ID NO: 57 - DNA, 3121 bp, Toxoplasma gondii
  source 1..3121: mol_type = unassigned DNA
ttttctgtgcatctgtgctgcaaaacgggcctctgtgcattatttccccaccaacaattgccgcgtcgatccgggtcccgctcaagctctgcagaactaggctctcgatatagatcagtacaatcattcgcttctgacaatcgcatcgactgagcgacgcgttgatcgtcgactgtcgtgcgtcgcattcgggcatctcgaaccggtgttgattccctgtgtcattatttcacttccgtccttctctcgtggcgatctataatacgcgtgtgttgttgcgtgcattgcttgtgttgttgtggatgtgttttcttttgtgaccgctcacgaacaccccacgcaaaatgagagcgtcgctcccggttcacctcgttgtgtgcacgcagctaagtgccgtttggtttggagtggctaaagcccatggtggacaccgactggaaccgcatgttcccggattcctgcaaggcttcactgatatcacgcctgcaggtgatgacgttagtgccaacgtaacaagttcggagcctgcaaaacttgatctctcttgtgtgcactctgacaataagggatcaagggctcccacaataggcgagccagtgccagatgtgtccctggaacaatgtgctgcgcaatgcaaggctgttgatggctgcacacatttcacttataatgacgattcgaagatgtgccatgtgaaggagggaaaacccgatttatacgatctcacaggaggcaaaacagcatcgcgcagttgcgatagatcatgcttcgaacaacacgtatcgtatgagggagctcctgacgtgatgacagcgatggtcacgagccagtcagcggactgtcaggctgcgtgtgcggctgacccgagctgcgagatcttcacttataacgaacacgaccagaaatgtactttcaaaggaagggggttttctgcgtttaaggaacgaggggtgttgggtgtgacttccgggccgaaacagttctgcgatgaaggcggtaaattaactcaagaggagatggaagatcagatcagtggctgcattcaattgagtgacgttggatcaatgactgctgacctggaggagcctatggaggctgattctgttggcgcttgtatggaacggtgccgctgtgatggaagatgcacgcacttcacgttcaacgataatactcggatgtgctacctcaaaggtgacaagatgcagttgtactcatctccaggtgacagaaccggcccaaagagctgcgattcaagctgcttctcgaacggggtttcttacgtcgatgatccggcgacagatgttgagaccgtattcgaaatttcacacccaatttattgtcaagtaatctgcgccgcaaatccgttgtgtacagtgtttcagtggtatgcctccgaggcaaagtgcgtcgtcaagagaaaggggttttacaaacacagaaaaacaggtgtcacgggagtcacagtgggccctcgggagttctgcgattttggcggtagcatccgcgaccgagaagaggcagacgccgttggatcagacgatggcctcaacgcggaagcaactatggcaaattctcctgattttcacgacgaagtagaatgcgtccacacgggcaacattgggtcaaaagcacaaaccattggagaagtgaaacgcgcaagtagtttgagtgagtgcagagccagatgccaagcggagaaagaatgcagccactacacttacaatgtaaaatccggtttgtgttatccaaaaagaggaaagcctcaattttataagtatcttggcgacatgacgggatccagaacatgtgatacaagttgccttaggaggggagtcgattactcacagggccctgaagtaggaaagccttggtattctacgctgccgacagactgccaagttgcatgcgacgctgaggatgcttgcctggtgttcacctgggattcggcgacgtcacgatgctacctcatcggctcaggtttctcggcacatcgacggaacgacgtggatggcgtggtatctggaccctatactttctgtgacaatggcgaaaaccttcaggtgcttgaagcgaaagacacagaatgacccaggagggtgccagatactttgtgtgactgcgacatgcagtcatgtactcaaagtgttgtacatggacaggaggactttttttttaagtcattgcagaggtgcgttttcggagcagcactataactgcgtcagcgactaagcacgccacgtagctgaatgaaacgcagccaccttcgtgtatgtatgcttcgttttttgtcgctgtgcagttttgaatcatttcccttatgggacatttctgaaaaatgctccccgttcgcttgtagcactatgagaggggccgaagactgcaatggaggtagcgctgcgttgaaaagacgaggcgctacatttcgcgtagcgacaaggccgtgtagagttttgcttttcgcgagacactgctctgagtgtcatatgcatcaaatgcagtggtagcacacagaggtgagaagaatgatcacctgcgggggaatggctttgctaaacaacaaggtcgctgtgtgactttacacaacgaaactactgtggtgagtgctcagttgagtgaaaagaaatgccgcgttatcgtgagttctggttcggtggactttgccaccgtagtaaaactcaacctgtaacggaatgcccagttttactgctctctttaaagggcgtccacgttctctatattcaagctgtttacccacctgcgtttcggtgcatcgcgcgtgccacatcaaaaatccaggtaacggtgcgggacctatgctacactttatatctctcagaaagcatacacccactgattatggacaacgctgtggtcgcgttgtaccacaatgcaggaatactcagttcaccttgcaagtgttctggtgttcattgcgtgtcagaagtacacgaaaagagacttctttggcctccaagtgatacgtaaccgcggcagtcatgaacagagtcactcgtgcttctgaaacgcacgtcttctgtacagagacagatgcagtgtgcatacaggaagcccctcgattgttgccgtagcaggtagccagtagaagaaacaaagacacggt

SEQ ID NO: 58 - DNA, 54 bp, Toxoplasma gondii
  source 1..54: mol_type = unassigned DNA
actgtggtctctaggtatgagttcggagcctgcaaaacttgatctctcttgtgt

SEQ ID NO: 59 - DNA, 47 bp, Toxoplasma gondii
  source 1..47: mol_type = unassigned DNA
actgttctagatcagccactgatatgatcttccatctcctcttgagt

SEQ ID NO: 60 - protein, 349 aa, Toxoplasma gondii
MRLFRCCAAAVVAAESLLWLKNGSPFFAFLPGNGEIADNCSGNPCGGTAAGTCINTPSGYDCRCEPGYVLGVENDQVTCMMPSGVPMANFVQLSEKPAACSSNPCGPEAAGTCNETNSGYICRCNQGYRISLDGTGNVTCIVRQESGCEENGCGPPDAVQSCRRLTGTAGRLCVCKENFIATIDASAHITCKRVPPHYRKPPFEFGKGGHPVDSEPSKRQREDEGESREPESDSTEPGRDQERRTPLEESQEPEGSTPDSQQSRGGSGSDSTESEEQGKEREEGSGHAGAIAGGVIGGLLLLSAAGAGVAYMRKSGSGGGEEIEYERGIEAAEASEVEVLVDLDSKTWD

SEQ ID NO: 61 - protein, 326 aa, Toxoplasma gondii
SPFFAFLPGNGEIADNCSGNPCGGTAAGTCINTPSGYDCRCEPGYVLGVENDQVTCMMPSGVPMANFVQLSEKPAACSSNPCGPEAAGTCNETNSGYICRCNQGYRISLDGTGNVTCIVRQESGCEENGCGPPDAVQSCRRLTGTAGRLCVCKENFIATIDASAHITCKRVPPHYRKPPFEFGKGGHPVDSEPSKRQREDEGESREPESDSTEPGRDQERRTPLEESQEPEGSTPDSQQSRGGSGSDSTESEEQGKEREEGSGHAGAIAGGVIGGLLLLSAAGAGVAYMRKSGSGGGEEIEYERGIEAAEASEVEVLVDLDSKTWD

SEQ ID NO: 62 - DNA, 3394 bp, Toxoplasma gondii
  source 1..3394: mol_type = unassigned DNA
cagtccggagcacactcctacaataaacttgatacgtgtcattttgtgaaacgacacagcacataaccactcggactgtctcacgaagctgtagggcggattcaccaatgatctttcgcagccgatccaaaactacttgcccacttccggtgtacgtacatcgcgcgacatgagaggcattcattgttttccatagaaaacactactggacaaccattcggtagcgcacaagttgagcctctgacaaatctttcctcatcacgtgaatacacgctgcgtgattcgtcagtgactccactgtggtctttaaccaccatcagagtcctgtaagcatcctttgtttccgtttaaaatgcctgccagatggcacgacgccgtctggttttgccggctttctccgagtcctattagactttgatgccttacggcttttttttaagaatggttcttttgagatttgccgactttccagttccgccaccagacgctcctgttgaactgccaccggcacgatgcagtattccgccacgaaaacgcgcaccgcaagctccgctaccattaaacgggtttcgtctgctttagatgtttccttccgcgtcatcaaggcaaaagcattgccactgatgttaccgaagctttcccgccatgctgcgcacaatgcccaatcttccgtcacggacctcttccggtaaccacctaaaggaggattactgggcaacccaaaacgctgcaacaagaagcacagtccaggtgtcgctagattcgagcctgcatggtcgttccgtagctccatacaacaattctctgtgtgacggcgagaggagtaacgcgctagtgtgtgtcagcgacgcggcagtcgatccgatcctgcaacaggcagaggtgtgtcgatgctcagtgatgcgacggcgtatctgaagaggactgtagctccaccacgaccttcgtgggagcacgaagtgtactctgttgtcgtcggtctcgtatttttttgagttgtgtacttcgctgcaagaggagggtgagattcgacatctgtgggcgtttgggatcgtgatgacatcgactgtgctttgatatatgatgtgttttttttcgattggatgagcacattccagtaagcttcctgccgcgcgtctctgctatgaggctcttccggtgctgtgctgcggccgttgtggcggccgaatcgttactgtggctgaagaacggctccccgttttttgcctttcttcctgggaatggagagattgcagacaactgctctgggaatccatgcggtggcaccgcagctggtacgtgcataaacacaccatctggatatgattgcaggtgcgaaccaggctacgttctgggcgttgaaaatgaccaggtcacgtgcatgatgccctcaggtgtacccatggctaattttgtacagctgtcggaaaagcctgcagcttgcagctcaaacccttgtggacctgaggcagccggcacctgcaacgagacaaacagtggttacatttgccgctgtaatcaaggctacagaatatctctcgacgggacaggaaacgtgacatgtattgtaagacaggaaagcggctgtgaggaaaacgggtgtgggccgccagatgcagtacagagttgccgccgactaacagggacggcaggtcgactatgtgtatgcaaggaaaactttatagcgacaatcgacgccagtgcccatatcacctgcaagcgtgtgcctccccattataggaagcctcccttcgaatttggcaagggaggtcatcctgtggactcagaaccatcgaaacgccagagggaagatgaaggtgaaagtcgtgagcctgaaagcgactcaacagaaccggggagagatcaggaaagaagaacaccacttgaggaaagccaggaaccggaaggaagcaccccggacagtcagcagagccgaggtggttctggtagcgacagtaccgagagcgaggaacaaggaaaggagagagaggaaggaagtggacatgctggtgcgatcgctgggggagttattggaggcctgttacttctgagcgctgccggagcgggtgttgcatacatgagaaagagtgggagcggtggaggggaggagatagaatacgagaggggtatcgaggctgcagaggccagtgaagtcgaagtcctcgttgatttggatagcaaaacatgggattaacacgttctcggctgagacttcacaatgtagggtgtcgctggcagatcagctgcaatgcgagaggtgacgcgagtagtgagcaccgcttcttttaagcgcggacattgtgctcggtcttctgtcacccccgaatcaaaacacatgtatgataatagttcctgttgacttcccctgccgacaaagaactgctgtgtcgaggccggcttctgtgcactcatcccaaatgagatggactgatgttttagagacacctcatcgccgacggaaaccatcagctcccagagaaactatgctgcgtcgttttttaggtgatctgttgcgtaatgcgcaccttcatatcatctgtgtgttgactgtttggtcgttttccgtttagtcaaatgaatgcagtgaaatgcagggaatttagcagacaccgagaactgtcctcttgttctgtgcgcgagttgtttttaacgtatagcgatgcgtttgcacttgatattaccctaagccatcagtgggtatttagaggagcccacaggtgatgggggtgatccctgtttcttgtcatttggcttgtagggttcgctggaactatctggtgtcacggaagagtggctttactgtctgtccccaaacgcaaggcatcagtgtaaccccgataggactctggagacttctgcttcactgccgcgttgcaattttcccgcgtcatgtggcaataacggtaattccacgtgcacgccgcataccggatctttgctcccaggctttcttatgaggtcggcatacgtacagcggcggcgtacctccgctctagagaagaccggtccaaccgactttgaacagcatgcttgtgaatgagtgcttaaacaccctgaagtgatggtggaatgtagcagtctgggacggttgatgcgaggatatcaccattagcatagactaccttgctctttagcgaggcgagacaacttatttaggtagccatgaaacacctcgatagtatcaatgacgacgtgcggttcaccaacttccgtcgctagcgcagaaaacagtcggaaacacaactcggtgagcacctgaagtgtcagtacacattcgaccgtcgggacccgggattccgcaagtggcacccgctggtccagtagcaggaacctagttcattcagtataacagatttggggcggcaaagagcaatttgctcgacctaacgcttgc

SEQ ID NO: 63 - DNA, 44 bp, Toxoplasma gondii
  source 1..44: mol_type = unassigned DNA
actgtgtgctctaggtatgtccccgttttttgcctttcttcctg

SEQ ID NO: 64 - DNA, 39 bp, Toxoplasma gondii
  source 1..39: mol_type = unassigned DNA
actgttctagattaatcccatgttttgctatccaaatca